SEQUENCE LISTING (18 sequences)

SEQ ID NO: 1 - protein, 1319 aa, Salmonid alphavirus subtype 3
MFPMQFTNSAYRQMEPMFAPGPRGQVQPYRPRTKRRQEPQVGNAAIAALANQMSALQLQVAGLAGQARVDRRGPRRVQKSKQKKKNPSNGEKPKEKKKKQKQQEKKGSGGVIKKPRNRPGKEVRISVKRARQSTFPVYHDGAISGYAVLIGSRVFKPAHVKGKIDHPELADIKFQVAEDMDLEAAAYPKSMRDQAAEPATMMDGVYNWEYGTIRVEDNVVIDASGRGKPGDSGRAITDNSGKVVGIVLGGGPDGRRTRLSVIGFDKKMKAREIAYSEAIPWTRAPALLLLPMVIACTYNSNTFDCSKPSCQDCCITAEPKKAMAMLKDNLNDPNYWDLLIAVTTCNSARKKRAVSTSPAAVYDTQILAAHAAASPYRAYCPDCDGTACISPIAIDEVVSSGSDHVLRIRVGSQSGVTAKGGAAGETSLRYLGRDGKVHAADNTRLVVRTTAKCDVLQATGHYILASCPEGQSITVAATLDGTRHQCTTVFEHQVTEKFTRERSKGHHLSDLTKKCTRFSTTPKKSAPYLVDVYDALPISVEISTVVTCNDNQCTVKVPPGTTVKFDKKCKSAAQATVTFTSDSQTFTCEEPVLTAASITQGKPHLRSSMLPSGGKEVKARIPFPFPPETATCRVSVAPLPSITYEESDVLLAGTAKYPVLLTTRNLGFHSNATSEWIQGKYLRRIPVTPQGIELTWGNNAPLHFWSSVRYASGDADAYPWELLVHHTKHHPEYAWAFVGVACGLLVIAVCMFACACNRVRYSLVANTFNPNPPPLTALTAALCCIPGARADQPYLDIIAYLWTNSKVAFGLQCAAPVACVLIVTYALRHCRLCCKSFLGVRGWSALLVILAYVQSCKSYEHTVVVPMDPRAPSYEAVINRNGYDPLKLTIAVNFTVISPTTALEYWTCASVPIVEPPHVGCCTSVSCPSDLSTLHAFTGKAVSDVHCDVHTNVYPLLWGAAHCFCSTENTQVSAVAATVSEFCAQDSERAEAFSVHSSSVTAEVLVTLGEVVTAVHVYVDGVTSARGTDLKIVAGPITTDYSPFDRKVVRIGEEVYNYDWPPYGAGRPGTFGDIQARSTNYVKPNDLYGDIGIEVLQPTNDHVHVAYTYTTSGLLRWLQDAPKPLSVTAPHGCKISANPLLALDCGVGAVPMSINIPDAKFTRKLKDPKPSALKCVVDSCEYGVDYGGAATITYEGHEAGKCGIHSLTPGVPLRTSVVEVVAGANTVKTTFSSPTPEVTLEVEICSAMVTCASECTPPKEHVVATRPRHGSDTGGYISGPAMRWAGGIVGTLAVLFLILAVTYCVVKKCRSKRIRIVKS

SEQ ID NO: 2 - DNA, 3960 bp, Salmonid alphavirus subtype 3
  source 1..3960: mol_type = other DNA
atgtttcccatgcaattcaccaactcagcctatcgccagatggagcccatgttcgcaccgggtcctcgaggacaagtacagccgtaccggccgcgcactaagcgtcgccaagagccgcaagtcggcaacgctgctattgctgccctcgcgaaccagatgagcgcgctccagctacaggtggctggacttgccggccaggcaagggtggatcgtcgtggaccaagacgtgttcagaaaagcaagcagaagaagaagaatccctccaacggagaaaaacccaaggagaagaagaagaagcaaaaacaacaggagaagaaagggagcggtggcgtaatcaaaaaaccacggaaccggcccggaaaggaggtaaggatctccgtaaagcgtgcccgacagagcaccttccccgtgtaccatgacggtgctatatccggctacgctgtgctgattggttctcgcgtgtttaagccagcgcacgtgaagggtaagatcgaccaccccgagctggcggacatcaagttccaggtcgccgaggacatggacctcgaagcagctgcttaccctaagagtatgcgagaccaggcggctgaaccagcaaccatgatggacggagtgtataactgggagtacggcactatcagagtggaggacaatgtcgtaattgacgcgagcggcagaggtaagccgggtgacagtggaagggccattaccgacaactcaggaaaggttgttggcattgtcctcggagggggacccgacggcaggcgcacacgcctctccgtgataggtttcgacaagaagatgaaggctagagagatcgcctacagcgaggccataccttggacacgtgctccagccctcctgctgctgcccatggtcatcgcctgtacctacaactccaacacctttgattgctccaaaccgtcctgccaggactgttgtatcactgctgaaccaaagaaggccatggctatgttgaaggacaacctgaacgacccgaactattgggacctactcattgccgtcaccacctgcaattccgcccggaaaaagagggctgtgtctacgtcgcctgccgccgtttacgacacacaaatcctcgccgcccacgcagctgcctccccgtacagggcgtattgccccgactgtgatggaactgcctgcatctcgccgatagctattgacgaggtggtaagcagcggtagcgaccacgtcctccgcatccgggtcggttctcaatcgggagtgaccgctaaaggcggtgcggcgggtgaaacatctctgcgatacctgggaagggacggtaaggtgcacgccgcggacaacacgcggcttgtggtgcgcaccactgcaaagtgcgatgtgctgcaggccaccggccactacatcctggccagctgcccagaggggcagagtattactgttgcggccacactggacggcacccgccaccaatgcaccacggttttcgaacatcaagtaacggagaagttcaccagagaacgcagcaagggccaccacttgtccgacctgaccaagaagtgcaccagattttccaccaccccgaagaagtccgccccctacctcgttgacgtgtacgacgctctgccgatttctgtagagattagcaccgttgtaacatgcaacgacaatcagtgcacagtgaaggtgccacccggtaccacagtgaaattcgataagaagtgcaagagcgctgcccaagcgaccgttacctttaccagcgactcccagacgtttacgtgtgaggagccggttctgacggccgccagtatcacccagggcaagccgcaccttagatcatctatgttgcccagcggaggcaaggaagtgaaggcgaggatcccattcccgttcccgccagagaccgcgacctgcagagtaagtgtcgccccgctgccgtcgatcacctatgaggaaagcgacgttctgctggccggtaccgcgaagtaccccgtgctgctgactacacggaaccttggtttccacagcaatgccacatccgaatggatccagggtaagtacttgcgccgtatcccggtcacgccccaagggatcgaactaacgtggggaaataacgcaccgttgcacttctggtcatctgttaggtacgcatctggggacgccgacgcgtacccttgggaacttctggtgcaccacaccaagcaccatccggagtacgcgtgggcgtttgtaggagttgcatgtggtctgctggttattgcagtatgcatgttcgcgtgcgcatgcaacagagtgcggtactctttggtcgccaacacgttcaacccgaacccaccaccactgaccgcactgactgcagcattgtgctgcatacctggggctcgtgcggaccaaccctacctggacatcattgcctacctgtggaccaacagcaaagtggccttcgggctgcaatgcgcggcgcccgtggcttgcgtgctcatcgtcacatacgcccttagacactgcagactgtgctgcaagtcttttttaggggtaagagggtggtcagctctgctggtcatccttgcgtatgtacagagctgcaagagctacgaacacaccgtggtggtcccaatggacccgagagccccgtcgtacgaagcagtgataaaccggaatgggtatgatcccttgaagctgaccattgcagtgaactttaccgtcatctcaccaactacagctctggaatactggacttgcgcaagtgtccctatcgtcgagccgccccatgtgggctgctgcacgtcggtgtcctgcccctccgacctctccacactgcacgcgttcaccggcaaagccgtctccgacgtgcactgcgatgtgcacacaaacgtgtaccccttgttgtggggtgcggctcattgcttctgctccaccgagaatacacaagtcagcgcggtggctgccaccgtctctgaattttgtgctcaggactcagagcgcgccgaagcgtttagcgttcacagcagctcagtcactgcagaggttctggtgacgcttggtgaagtggtgacggcagtccacgtttacgtggacggggtaacatctgccaggggtaccgacctcaagatcgtggctgggccaataacaactgactactccccgttcgatcgcaaagtagtccgtatcggcgaagaggtctataactacgactggcctccttacggggctggtcgaccaggcacattcggggatattcaagctaggtcaaccaactatgtcaagcccaatgatctgtacggggatatcggaatcgaagtactgcagccgactaatgaccatgtgcacgtggcttacacgtatacgacctccgggttactgcgttggctgcaggacgctccgaagccacttagtgtcacagcaccgcacggttgtaagatcagtgccaacccgctcctggccctcgattgtggggttggcgccgtccccatgtccatcaacattccggacgcgaagttcacccgcaaattgaaggatccgaaaccatcggccctgaaatgcgtggtggacagttgcgagtacggggtggactacgggggcgccgccacgatcacctatgagggccacgaggctgggaagtgcgggatccattccctgacaccaggagtccctctgagaacatcagtggtcgaagtggttgccggcgctaacaccgtcaaaacgaccttctcctcacccacgcccgaggtcacactcgaggtagagatctgttcggcaatggtgacgtgcgccagtgagtgcactccaccgaaggaacatgtagtcgcaaccaggcctcgccatggcagcgacactggaggctacatctccgggcccgcaatgcgctgggccggtgggattgtagggaccttagcggtcctgttcctcatccttgccgttacctactgcgtggtgaagaagtgccgctctaaaagaatccggatagtcaagagctaa

SEQ ID NO: 3 - DNA, 3960 bp, Synthetic construct
  source 1..3960: mol_type = other DNA
atgttccccatgcagttcactaatagcgcatacagacaaatggagcccatgtttgctcccggaccaagagggcaggttcaaccttatagacctcggactaagaggcgacaggagcctcaagttggcaatgctgccatcgctgccttggcgaaccagatgtctgctttacagctgcaggtggcagggctagctggtcaggcccgagtcgaccgaagaggacctcggagagtgcagaaatccaagcaaaagaaaaaaaatccatctaatggggagaaacctaaggagaagaagaaaaaacagaagcagcaggaaaagaagggttcaggaggtgttattaaaaagcctcgtaatcggcccgggaaagaggtgcgaattagtgtgaagcgtgcaagacagagcacctttccagtttatcatgacggagcaatttcgggctacgctgtgctgatcgggtcaagggtgttcaaaccagcccacgttaaaggaaaaatcgatcatccggagcttgctgacatcaagtttcaggtcgccgaagacatggacctggaggcagctgcctatcccaagtcgatgcgagatcaagccgctgaacccgcgaccatgatggatggtgtctacaactgggaatatgggacgatccgagtggaggataacgtggttattgacgcatccggccggggcaaaccgggggactctggacgcgccattaccgacaactcaggtaaagtggtgggcatcgtgctcggcggaggtcccgatggccgcaggacccgattatccgttatcggattcgacaagaagatgaaggcaagggagattgcatatagcgaagcaattccctggactcgtgctcctgctctgctgcttcttccaatggtcatcgcatgtacctataactccaacacctttgactgttcaaagcccagttgccaagattgctgtatcaccgcagaaccgaagaaggctatggccatgctgaaagacaatctgaatgacccaaattactgggacctgctcatcgcggtgaccacttgcaactctgctaggaaaaagagagccgtgtccacatctccagcagctgtgtatgacactcagattctggccgcacacgcagcggcctcgccataccgagcttactgccctgattgtgatggtactgcttgcatttctcccattgcaattgatgaggttgtgtcaagcggctctgaccacgtgctccgcattcgtgtgggctcacagtctggagtgactgccaaaggaggtgctgcaggagagacatccttgcgttatctgggccgggatggaaaagtccacgcagcagataatacaaggttggtcgtgcgcaccacagccaaatgtgacgtgttacaagctacaggacactacatcctggcatcctgtcctgaaggacaaagcattacagtggccgctaccctagacggaactcgtcatcagtgcactacagtgtttgaacatcaggtcacagaaaagttcacgagagaaaggtcaaagggtcaccacctgtcagacttaacgaaaaagtgcacccgttttagcaccacaccaaagaaatctgcaccgtatcttgtggatgtttacgacgcgctgccgatctcagtggaaatctccacggtggtcacatgcaatgataaccaatgtaccgtcaaagtgcctccagggaccactgttaagtttgacaaaaaatgtaagtccgctgcacaggcgactgtgactttcactagtgattcccagacattcgcatgtgaagaacctgtactcacggctgccagtatcacacaaggaaaacctcatcttcgcagtagcatgttgcccagcggggggaaagaggtgaaagctagaatccccttcccatttccaccagagacggcgacgtgtcgtgtgagtgtggccccactaccaagtataacctacgaggagtctgatgttctcctggccggcacggccaagtaccctgttctcttaaccacaagaaacctgggcttccactctaacgccacttctgagtggattcaaggcaaataccttcgacgcattccagttacccctcaggggatagagctgacctggggcaacaacgcgcctctgcacttttggagttctgttagatatgcctcgggcgatgctgatgcatatccatgggagttgttagtgcatcacacaaaacaccatccagagtacgcctgggcattcgtaggcgttgcctgtggtcttctcgtcatagccgtctgcatgtttgcctgcgcttgtaaccgggtgaggtacagtctggtggccaataccttcaaccctaaccctcctcccctgacagcgctgactgcggcactttgctgcatccccggagctcgagcagatcagccatacttggatataatcgcgtacctgtggactaactcgaaagttgcatttggcctccagtgtgctgctcctgtggcctgtgttctaatagtgacctatgccctgcgccactgccgcttgtgttgcaagagttttcttggcgtaagaggatggtccgcactccttgtgatcctggcgtacgtgcagtcttgtaaaagctacgaacacaccgtggttgttccaatggacccgagagccccatcctatgaggctgtgattaacaggaatggttatgatcctctgaagctgaccatcgcagtcaatttcacagtcatatcaccaactacggctctggagtactggacatgcgctagtgtcccaattgtggagccgccccatgttggctgctgtacatcagttagctgcccgtctgatctatctacactccacgcttttaccggaaaagctgtaagcgatgtgcattgtgacgtacacactaatgtatacccactgctgtggggtgctgcacactgtttctgctccactgaaaacactcaagtctcagctgttgcagcaacagtttccgagttctgtgcccaggacagcgagagagctgaggccttttccgtccattcatctagcgtcacagcggaggttttggtaacgctgggtgaggttgtcactgcagtgcacgtctacgtggatggggtaacgtctgctagaggtacagacctcaagatcgttgccggacccataacaaccgattacagcccgtttgatagaaaggtcgttagaattggtgaggaggtatataactatgactggcctccttacggcgcgggacggccgggcacatttggagatatccaggctcggtcaaccaactacgtaaagccaaacgacctctatggagacataggaattgaagtgttgcagcccaccaatgatcacgtacatgtcgcttacacctacacgacgagtggtctgctacgctggctgcaggatgcccctaaaccgctctctgtcacagccccccatgggtgcaaaattagcgccaatcctcttctggccctggactgcggggtgggagcggtgccgatgagcatcaacatcccagacgcaaaattcacgcgtaaattgaaagatccaaagccttcagccctgaagtgtgtcgtcgacagttgtgagtatggtgttgattatggaggcgcagccactattacatatgagggtcatgaagccgggaagtgtggaatacacagtctcactcctggggtacctctccgcacctcagtcgtggaagttgtagcaggagccaacacagtcaaaactacattctcatcacccacacctgaggtgactcttgaggtcgagatctgctctgccatggtgacctgtgcaagtgaatgcacacccccaaaagaacatgttgtggctactaggcccagacatggctcggacaccggaggatacatcagcggtccagcgatgaggtgggccggcgggattgtcggaacactggctgtgttgttcctgatccttgcagtaacatactgtgtggtgaagaagtgcaggagcaaaaggatcagaatagtgaaatcgtaa

SEQ ID NO: 4 - protein, 281 aa, Salmonid alphavirus subtype 3
MFPMQFTNSAYRQMEPMFAPGPRGQVQPYRPRTKRRQEPQVGNAAIAALANQMSALQLQVAGLAGQARVDRRGPRRVQKSKQKKKNPSNGEKPKEKKKKQKQQEKKGSGGVIKKPRNRPGKEVRISVKRARQSTFPVYHDGAISGYAVLIGSRVFKPAHVKGKIDHPELADIKFQVAEDMDLEAAAYPKSMRDQAAEPATMMDGVYNWEYGTIRVEDNVVIDASGRGKPGDSGRAITDNSGKVVGIVLGGGPDGRRTRLSVIGFDKKMKAREIAYSEAIPW

SEQ ID NO: 5 - DNA, 843 bp, Synthetic construct
  source 1..843: mol_type = other DNA
atgttccccatgcagttcactaatagcgcatacagacaaatggagcccatgtttgctcccggaccaagagggcaggttcaaccttatagacctcggactaagaggcgacaggagcctcaagttggcaatgctgccatcgctgccttggcgaaccagatgtctgctttacagctgcaggtggcagggctagctggtcaggcccgagtcgaccgaagaggacctcggagagtgcagaaatccaagcaaaagaaaaaaaatccatctaatggggagaaacctaaggagaagaagaaaaaacagaagcagcaggaaaagaagggttcaggaggtgttattaaaaagcctcgtaatcggcccgggaaagaggtgcgaattagtgtgaagcgtgcaagacagagcacctttccagtttatcatgacggagcaatttcgggctacgctgtgctgatcgggtcaagggtgttcaaaccagcccacgttaaaggaaaaatcgatcatccggagcttgctgacatcaagtttcaggtcgccgaagacatggacctggaggcagctgcctatcccaagtcgatgcgagatcaagccgctgaacccgcgaccatgatggatggtgtctacaactgggaatatgggacgatccgagtggaggataacgtggttattgacgcatccggccggggcaaaccgggggactctggacgcgccattaccgacaactcaggtaaagtggtgggcatcgtgctcggcggaggtcccgatggccgcaggacccgattatccgttatcggattcgacaagaagatgaaggcaagggagattgcatatagcgaagcaattccctgg

SEQ ID NO: 6 - protein, 71 aa, Salmonid alphavirus subtype 3
TRAPALLLLPMVIACTYNSNTFDCSKPSCQDCCITAEPKKAMAMLKDNLNDPNYWDLLIAVTTCNSARKKR

SEQ ID NO: 7 - DNA, 213 bp, Synthetic construct
  source 1..213: mol_type = other DNA
actcgtgctcctgctctgctgcttcttccaatggtcatcgcatgtacctataactccaacacctttgactgttcaaagcccagttgccaagattgctgtatcaccgcagaaccgaagaaggctatggccatgctgaaagacaatctgaatgacccaaattactgggacctgctcatcgcggtgaccacttgcaactctgctaggaaaaagaga

SEQ ID NO: 8 - protein, 438 aa, Salmonid alphavirus subtype 3
AVSTSPAAVYDTQILAAHAAASPYRAYCPDCDGTACISPIAIDEVVSSGSDHVLRIRVGSQSGVTAKGGAAGETSLRYLGRDGKVHAADNTRLVVRTTAKCDVLQATGHYILASCPEGQSITVAATLDGTRHQCTTVFEHQVTEKFTRERSKGHHLSDLTKKCTRFSTTPKKSAPYLVDVYDALPISVEISTVVTCNDNQCTVKVPPGTTVKFDKKCKSAAQATVTFTSDSQTFACEEPVLTAASITQGKPHLRSSMLPSGGKEVKARIPFPFPPETATCRVSVAPLPSITYEESDVLLAGTAKYPVLLTTRNLGFHSNATSEWIQGKYLRRIPVTPQGIELTWGNNAPLHFWSSVRYASGDADAYPWELLVHHTKHHPEYAWAFVGVACGLLVIAVCMFACACNRVRYSLVANTFNPNPPPLTALTAALCCIPGARA

SEQ ID NO: 9 - DNA, 1314 bp, Synthetic construct
  source 1..1314: mol_type = other DNA
gccgtgtccacatctccagcagctgtgtatgacactcagattctggccgcacacgcagcggcctcgccataccgagcttactgccctgattgtgatggtactgcttgcatttctcccattgcaattgatgaggttgtgtcaagcggctctgaccacgtgctccgcattcgtgtgggctcacagtctggagtgactgccaaaggaggtgctgcaggagagacatccttgcgttatctgggccgggatggaaaagtccacgcagcagataatacaaggttggtcgtgcgcaccacagccaaatgtgacgtgttacaagctacaggacactacatcctggcatcctgtcctgaaggacaaagcattacagtggccgctaccctagacggaactcgtcatcagtgcactacagtgtttgaacatcaggtcacagaaaagttcacgagagaaaggtcaaagggtcaccacctgtcagacttaacgaaaaagtgcacccgttttagcaccacaccaaagaaatctgcaccgtatcttgtggatgtttacgacgcgctgccgatctcagtggaaatctccacggtggtcacatgcaatgataaccaatgtaccgtcaaagtgcctccagggaccactgttaagtttgacaaaaaatgtaagtccgctgcacaggcgactgtgactttcactagtgattcccagacattcgcatgtgaagaacctgtactcacggctgccagtatcacacaaggaaaacctcatcttcgcagtagcatgttgcccagcggggggaaagaggtgaaagctagaatccccttcccatttccaccagagacggcgacgtgtcgtgtgagtgtggccccactaccaagtataacctacgaggagtctgatgttctcctggccggcacggccaagtaccctgttctcttaaccacaagaaacctgggcttccactctaacgccacttctgagtggattcaaggcaaataccttcgacgcattccagttacccctcaggggatagagctgacctggggcaacaacgcgcctctgcacttttggagttctgttagatatgcctcgggcgatgctgatgcatatccatgggagttgttagtgcatcacacaaaacaccatccagagtacgcctgggcattcgtaggcgttgcctgtggtcttctcgtcatagccgtctgcatgtttgcctgcgcttgtaaccgggtgaggtacagtctggtggccaataccttcaaccctaaccctcctcccctgacagcgctgactgcggcactttgctgcatccccggagctcgagca

SEQ ID NO: 10 - protein, 68 aa, Salmonid alphavirus subtype 3
DQPYLDIIAYLWTNSKVAFGLQCAAPVACVLIVTYALRHCRLCCKSFLGVRGWSALLVILAYVQSCKS

SEQ ID NO: 11 - DNA, 204 bp, Synthetic construct
  source 1..204: mol_type = other DNA
gatcagccatacttggatataatcgcgtacctgtggactaactcgaaagttgcatttggcctccagtgtgctgctcctgtggcctgtgttctaatagtgacctatgccctgcgccactgccgcttgtgttgcaagagttttcttggcgtaagaggatggtccgcactccttgtgatcctggcgtacgtgcagtcttgtaaaagc

SEQ ID NO: 12 - protein, 461 aa, Salmonid alphavirus subtype 3
YEHTVVVPMDPRAPSYEAVINRNGYDPLKLTIAVNFTVISPTTALEYWTCASVPIVEPPHVGCCTSVSCPSDLSTLHAFTGKAVSDVHCDVHTNVYPLLWGAAHCFCSTENTQVSAVAATVSEFCAQDSERAEAFSVHSSSVTAEVLVTLGEVVTAVHVYVDGVTSARGTDLKIVAGPITTDYSPFDRKVVRIGEEVYNYDWPPYGAGRPGTFGDIQARSTNYVKPNDLYGDIGIEVLQPTNDHVHVAYTYTTSGLLRWLQDAPKPLSVTAPHGCKISANPLLALDCGVGAVPMSINIPDAKFTRKLKDPKPSALKCVVDSCEYGVDYGGAATITYEGHEAGKCGIHSLTPGVPLRTSVVEVVAGANTVKTTFSSPTPEVTLEVEICSAMVTCASECTPPKEHVVATRPRHGSDTGGYISGPAMRWAGGIVGTLAVLFLILAVTYCVVKKCRSKRIRIVKS

SEQ ID NO: 13 - DNA, 1383 bp, Synthetic construct
  source 1..1383: mol_type = other DNA
tacgaacacaccgtggttgttccaatggacccgagagccccatcctatgaggctgtgattaacaggaatggttatgatcctctgaagctgaccatcgcagtcaatttcacagtcatatcaccaactacggctctggagtactggacatgcgctagtgtcccaattgtggagccgccccatgttggctgctgtacatcagttagctgcccgtctgatctatctacactccacgcttttaccggaaaagctgtaagcgatgtgcattgtgacgtacacactaatgtatacccactgctgtggggtgctgcacactgtttctgctccactgaaaacactcaagtctcagctgttgcagcaacagtttccgagttctgtgcccaggacagcgagagagctgaggccttttccgtccattcatctagcgtcacagcggaggttttggtaacgctgggtgaggttgtcactgcagtgcacgtctacgtggatggggtaacgtctgctagaggtacagacctcaagatcgttgccggacccataacaaccgattacagcccgtttgatagaaaggtcgttagaattggtgaggaggtatataactatgactggcctccttacggcgcgggacggccgggcacatttggagatatccaggctcggtcaaccaactacgtaaagccaaacgacctctatggagacataggaattgaagtgttgcagcccaccaatgatcacgtacatgtcgcttacacctacacgacgagtggtctgctacgctggctgcaggatgcccctaaaccgctctctgtcacagccccccatgggtgcaaaattagcgccaatcctcttctggccctggactgcggggtgggagcggtgccgatgagcatcaacatcccagacgcaaaattcacgcgtaaattgaaagatccaaagccttcagccctgaagtgtgtcgtcgacagttgtgagtatggtgttgattatggaggcgcagccactattacatatgagggtcatgaagccgggaagtgtggaatacacagtctcactcctggggtacctctccgcacctcagtcgtggaagttgtagcaggagccaacacagtcaaaactacattctcatcacccacacctgaggtgactcttgaggtcgagatctgctctgccatggtgacctgtgcaagtgaatgcacacccccaaaagaacatgttgtggctactaggcccagacatggctcggacaccggaggatacatcagcggtccagcgatgaggtgggccggcgggattgtcggaacactggctgtgttgttcctgatccttgcagtaacatactgtgtggtgaagaagtgcaggagcaaaaggatcagaatagtgaaatcg

SEQ ID NO: 14 - DNA, 4865 bp, Synthetic construct
  source 1..4865: mol_type = other DNA
gcgtataatggactattgtgtgctgatatgtacacttaagtagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagaagcttgccgccaccatgtttcccatgcaattcaccaactcagcctatcgccagatggagcccatgttcgcaccgggtcctcgaggacaagtacagccgtaccggccgcgcactaagcgtcgccaagagccgcaagtcggcaacgctgctattgctgccctcgcgaaccagatgagcgcgctccagctacaggtggctggacttgccggccaggcaagggtggatcgtcgtggaccaagacgtgttcagaaaagcaagcagaagaagaagaatccctccaacggagaaaaacccaaggagaagaagaagaagcaaaaacaacaggagaagaaagggagcggtggcgtaatcaaaaaaccacggaaccggcccggaaaggaggtaaggatctccgtaaagcgtgcccgacagagcaccttccccgtgtaccatgacggtgctatatccggctacgctgtgctgattggttctcgcgtgtttaagccagcgcacgtgaagggtaagatcgaccaccccgagctggcggacatcaagttccaggtcgccgaggacatggacctcgaagcagctgcttaccctaagagtatgcgagaccaggcggctgaaccagcaaccatgatggacggagtgtataactgggagtacggcactatcagagtggaggacaatgtcgtaattgacgcgagcggcagaggtaagccgggtgacagtggaagggccattaccgacaactcaggaaaggttgttggcattgtcctcggagggggacccgacggcaggcgcacacgcctctccgtgataggtttcgacaagaagatgaaggctagagagatcgcctacagcgaggccataccttggacacgtgctccagccctcctgctgctgcccatggtcatcgcctgtacctacaactccaacacctttgattgctccaaaccgtcctgccaggactgttgtatcactgctgaaccaaagaaggccatggctatgttgaaggacaacctgaacgacccgaactattgggacctactcattgccgtcaccacctgcaattccgcccggaaaaagagggctgtgtctacgtcgcctgccgccgtttacgacacacaaatcctcgccgcccacgcagctgcctccccgtacagggcgtattgccccgactgtgatggaactgcctgcatctcgccgatagctattgacgaggtggtaagcagcggtagcgaccacgtcctccgcatccgggtcggttctcaatcgggagtgaccgctaaaggcggtgcggcgggtgaaacatctctgcgatacctgggaagggacggtaaggtgcacgccgcggacaacacgcggcttgtggtgcgcaccactgcaaagtgcgatgtgctgcaggccaccggccactacatcctggccagctgcccagaggggcagagtattactgttgcggccacactggacggcacccgccaccaatgcaccacggttttcgaacatcaagtaacggagaagttcaccagagaacgcagcaagggccaccacttgtccgacctgaccaagaagtgcaccagattttccaccaccccgaagaagtccgccccctacctcgttgacgtgtacgacgctctgccgatttctgtagagattagcaccgttgtaacatgcaacgacaatcagtgcacagtgaaggtgccacccggtaccacagtgaaattcgataagaagtgcaagagcgctgcccaagcgaccgttacctttaccagcgactcccagacgtttgcgtgtgaggagccggttctgacggccgccagtatcacccagggcaagccgcaccttagatcatctatgttgcccagcggaggcaaggaagtgaaggcgagaatcccattcccgttcccgccagagaccgcgacctgcagagtaagtgtcgccccgctgccgtcgatcacctatgaggaaagcgacgttctgctggccggtaccgcgaagtaccccgtgctgctgactacacggaaccttggtttccacagcaatgccacatccgaatggattcagggtaagtacttgcgccgtatcccggtcacgccccaagggatcgaactaacgtggggaaataacgcaccgttgcacttctggtcatctgttaggtacgcatctggggacgccgacgcgtacccttgggaacttctggtgcaccacaccaagcaccatccggagtacgcgtgggcgtttgtaggagttgcatgtggtctgctggttattgcagtatgcatgttcgcgtgcgcatgcaacagagtgcggtactctttggtcgccaacacgttcaacccgaacccaccaccactgaccgcactgactgcagcattgtgctgcatacctggggctcgtgcggaccaaccctacctggacatcattgcctacctgtggaccaacagcaaagtggccttcgggctgcaatgcgcggcgcccgtggcttgcgtgctcatcgtcacatacgcccttagacactgcagactgtgctgcaagtcttttttaggggtaagagggtggtcagctctgctggtcatccttgcgtatgtacagagctgcaagagctacgaacacaccgtggtggtcccaatggacccgagagccccgtcgtacgaagcagtgataaaccggaatgggtatgatcccttgaagctgaccattgcagtgaactttaccgtcatctcaccaactacagctctggaatactggacttgcgcaagtgtccctatcgtcgagccgccccatgtgggctgctgcacgtcggtgtcctgcccctccgacctctccacactgcacgcgttcaccggcaaagccgtctccgacgtgcactgcgatgtgcacacaaacgtgtaccccttgttgtggggtgcggctcattgcttctgctccaccgagaatacacaagtcagcgcggtggctgccaccgtctctgaattttgtgctcaggactcagagcgcgccgaagcgtttagcgttcacagcagctcagtcactgcagaggttctggtgacgcttggtgaagtggtgacggcagtccacgtttacgtggacggggtaacatctgccaggggtaccgacctcaagatcgtggctgggccaataacaactgactactccccgttcgatcgcaaagtagtccgtatcggcgaagaggtctataactacgactggcctccttacggggctggtcgaccaggcacattcggggatattcaagctaggtcaaccaactatgtcaagcccaatgatctgtacggggatatcggaatcgaagtactgcagccgactaatgaccatgtgcacgtggcttacacgtatacgacctccgggttactgcgttggctgcaggacgctccgaagccacttagtgtcacagcaccgcacggttgtaagatcagtgccaacccgctcctggccctcgattgtggggttggcgccgtccccatgtccatcaacattccggacgcgaagttcacccgcaaattgaaggacccgaaaccatcggccctgaaatgcgtggtggacagttgcgagtacggggtggactacgggggcgccgccacgatcacctatgagggccacgaggctgggaagtgcggcatccattccctgacaccaggagtccctctgagaacatcagtggtcgaagtggttgccggcgctaacaccgtcaaaacgaccttctcctcacccacgcccgaggtcacactcgaggtagagatctgttcggcaatggtgacgtgcgccagtgagtgcactccaccgaaggaacatgtagtcgcaaccaggcctcgccatggcagcgacactggaggctacatctccgggcccgcaatgcgctgggccggtgggattgtagggaccttagcggtcctgttcctcatccttgccgttacctactgcgtggtgaagaagtgccgctctaaaagaatccggatagtcaagagctaagaattccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagctagcagatctactagttatcagcacacaattgcccattatacgc

SEQ ID NO: 15 - DNA, 4865 bp, Synthetic construct
  source 1..4865: mol_type = other DNA
gcgtataatggactattgtgtgctgatatgtacacttaagtagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagaagcttgccgccaccatgttccccatgcagttcactaatagcgcatacagacaaatggagcccatgtttgctcccggaccaagagggcaggttcaaccttatagacctcggactaagaggcgacaggagcctcaagttggcaatgctgccatcgctgccttggcgaaccagatgtctgctttacagctgcaggtggcagggctagctggtcaggcccgagtcgaccgaagaggacctcggagagtgcagaaatccaagcaaaagaaaaaaaatccatctaatggggagaaacctaaggagaagaagaaaaaacagaagcagcaggaaaagaagggttcaggaggtgttattaaaaagcctcgtaatcggcccgggaaagaggtgcgaattagtgtgaagcgtgcaagacagagcacctttccagtttatcatgacggagcaatttcgggctacgctgtgctgatcgggtcaagggtgttcaaaccagcccacgttaaaggaaaaatcgatcatccggagcttgctgacatcaagtttcaggtcgccgaagacatggacctggaggcagctgcctatcccaagtcgatgcgagatcaagccgctgaacccgcgaccatgatggatggtgtctacaactgggaatatgggacgatccgagtggaggataacgtggttattgacgcatccggccggggcaaaccgggggactctggacgcgccattaccgacaactcaggtaaagtggtgggcatcgtgctcggcggaggtcccgatggccgcaggacccgattatccgttatcggattcgacaagaagatgaaggcaagggagattgcatatagcgaagcaattccctggactcgtgctcctgctctgctgcttcttccaatggtcatcgcatgtacctataactccaacacctttgactgttcaaagcccagttgccaagattgctgtatcaccgcagaaccgaagaaggctatggccatgctgaaagacaatctgaatgacccaaattactgggacctgctcatcgcggtgaccacttgcaactctgctaggaaaaagagagccgtgtccacatctccagcagctgtgtatgacactcagattctggccgcacacgcagcggcctcgccataccgagcttactgccctgattgtgatggtactgcttgcatttctcccattgcaattgatgaggttgtgtcaagcggctctgaccacgtgctccgcattcgtgtgggctcacagtctggagtgactgccaaaggaggtgctgcaggagagacatccttgcgttatctgggccgggatggaaaagtccacgcagcagataatacaaggttggtcgtgcgcaccacagccaaatgtgacgtgttacaagctacaggacactacatcctggcatcctgtcctgaaggacaaagcattacagtggccgctaccctagacggaactcgtcatcagtgcactacagtgtttgaacatcaggtcacagaaaagttcacgagagaaaggtcaaagggtcaccacctgtcagacttaacgaaaaagtgcacccgttttagcaccacaccaaagaaatctgcaccgtatcttgtggatgtttacgacgcgctgccgatctcagtggaaatctccacggtggtcacatgcaatgataaccaatgtaccgtcaaagtgcctccagggaccactgttaagtttgacaaaaaatgtaagtccgctgcacaggcgactgtgactttcactagtgattcccagacattcgcatgtgaagaacctgtactcacggctgccagtatcacacaaggaaaacctcatcttcgcagtagcatgttgcccagcggggggaaagaggtgaaagctagaatccccttcccatttccaccagagacggcgacgtgtcgtgtgagtgtggccccactaccaagtataacctacgaggagtctgatgttctcctggccggcacggccaagtaccctgttctcttaaccacaagaaacctgggcttccactctaacgccacttctgagtggattcaaggcaaataccttcgacgcattccagttacccctcaggggatagagctgacctggggcaacaacgcgcctctgcacttttggagttctgttagatatgcctcgggcgatgctgatgcatatccatgggagttgttagtgcatcacacaaaacaccatccagagtacgcctgggcattcgtaggcgttgcctgtggtcttctcgtcatagccgtctgcatgtttgcctgcgcttgtaaccgggtgaggtacagtctggtggccaataccttcaaccctaaccctcctcccctgacagcgctgactgcggcactttgctgcatccccggagctcgagcagatcagccatacttggatataatcgcgtacctgtggactaactcgaaagttgcatttggcctccagtgtgctgctcctgtggcctgtgttctaatagtgacctatgccctgcgccactgccgcttgtgttgcaagagttttcttggcgtaagaggatggtccgcactccttgtgatcctggcgtacgtgcagtcttgtaaaagctacgaacacaccgtggttgttccaatggacccgagagccccatcctatgaggctgtgattaacaggaatggttatgatcctctgaagctgaccatcgcagtcaatttcacagtcatatcaccaactacggctctggagtactggacatgcgctagtgtcccaattgtggagccgccccatgttggctgctgtacatcagttagctgcccgtctgatctatctacactccacgcttttaccggaaaagctgtaagcgatgtgcattgtgacgtacacactaatgtatacccactgctgtggggtgctgcacactgtttctgctccactgaaaacactcaagtctcagctgttgcagcaacagtttccgagttctgtgcccaggacagcgagagagctgaggccttttccgtccattcatctagcgtcacagcggaggttttggtaacgctgggtgaggttgtcactgcagtgcacgtctacgtggatggggtaacgtctgctagaggtacagacctcaagatcgttgccggacccataacaaccgattacagcccgtttgatagaaaggtcgttagaattggtgaggaggtatataactatgactggcctccttacggcgcgggacggccgggcacatttggagatatccaggctcggtcaaccaactacgtaaagccaaacgacctctatggagacataggaattgaagtgttgcagcccaccaatgatcacgtacatgtcgcttacacctacacgacgagtggtctgctacgctggctgcaggatgcccctaaaccgctctctgtcacagccccccatgggtgcaaaattagcgccaatcctcttctggccctggactgcggggtgggagcggtgccgatgagcatcaacatcccagacgcaaaattcacgcgtaaattgaaagatccaaagccttcagccctgaagtgtgtcgtcgacagttgtgagtatggtgttgattatggaggcgcagccactattacatatgagggtcatgaagccgggaagtgtggaatacacagtctcactcctggggtacctctccgcacctcagtcgtggaagttgtagcaggagccaacacagtcaaaactacattctcatcacccacacctgaggtgactcttgaggtcgagatctgctctgccatggtgacctgtgcaagtgaatgcacacccccaaaagaacatgttgtggctactaggcccagacatggctcggacaccggaggatacatcagcggtccagcgatgaggtgggccggcgggattgtcggaacactggctgtgttgttcctgatccttgcagtaacatactgtgtggtgaagaagtgcaggagcaaaaggatcagaatagtgaaatcgtgagaattccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagctagcagatctactagttatcagcacacaattgcccattatacgc

SEQ ID NO: 16 - protein, 438 aa, Salmonid alphavirus subtype 3
AVSTSPAAVYDTQILAAHAAASPYRAYCPDCDGTACISPIAIDEVVSSGSDHVLRIRVGSQSGVTAKGGAAGETSLRYLGRDGKVHAADNTRLVVRTTAKCDVLQATGHYILASCPEGQSITVAATLDGTRHQCTTVFEHQVTEKFTRERSKGHHLSDLTKKCTRFSTTPKKSAPYLVDVYDALPISVEISTVVTCNDNQCTVKVPPGTTVKFDKKCKSAAQATVTFTSDSQTFTCEEPVLTAASITQGKPHLRSSMLPSGGKEVKARIPFPFPPETATCRVSVAPLPSITYEESDVLLAGTAKYPVLLTTRNLGFHSNATSEWIQGKYLRRIPVTPQGIELTWGNNAPLHFWSSVRYASGDADAYPWELLVHHTKHHPEYAWAFVGVACGLLVIAVCMFACACNRVRYSLVANTFNPNPPPLTALTAALCCIPGARA

SEQ ID NO: 17 - DNA, 1314 bp, Synthetic construct
  source 1..1314: mol_type = other DNA
gccgtgtccacatctccagcagctgtgtatgacactcagattctggccgcacacgcagcggcctcgccataccgagcttactgccctgattgtgatggtactgcttgcatttctcccattgcaattgatgaggttgtgtcaagcggctctgaccacgtgctccgcattcgtgtgggctcacagtctggagtgactgccaaaggaggtgctgcaggagagacatccttgcgttatctgggccgggatggaaaagtccacgcagcagataatacaaggttggtcgtgcgcaccacagccaaatgtgacgtgttacaagctacaggacactacatcctggcatcctgtcctgaaggacaaagcattacagtggccgctaccctagacggaactcgtcatcagtgcactacagtgtttgaacatcaggtcacagaaaagttcacgagagaaaggtcaaagggtcaccacctgtcagacttaacgaaaaagtgcacccgttttagcaccacaccaaagaaatctgcaccgtatcttgtggatgtttacgacgcgctgccgatctcagtggaaatctccacggtggtcacatgcaatgataaccaatgtaccgtcaaagtgcctccagggaccactgttaagtttgacaaaaaatgtaagtccgctgcacaggcgactgtgactttcactagtgattcccagacattcacatgtgaagaacctgtactcacggctgccagtatcacacaaggaaaacctcatcttcgcagtagcatgttgcccagcggggggaaagaggtgaaagctagaatccccttcccatttccaccagagacggcgacgtgtcgtgtgagtgtggccccactaccaagtataacctacgaggagtctgatgttctcctggccggcacggccaagtaccctgttctcttaaccacaagaaacctgggcttccactctaacgccacttctgagtggattcaaggcaaataccttcgacgcattccagttacccctcaggggatagagctgacctggggcaacaacgcgcctctgcacttttggagttctgttagatatgcctcgggcgatgctgatgcatatccatgggagttgttagtgcatcacacaaaacaccatccagagtacgcctgggcattcgtaggcgttgcctgtggtcttctcgtcatagccgtctgcatgtttgcctgcgcttgtaaccgggtgaggtacagtctggtggccaataccttcaaccctaaccctcctcccctgacagcgctgactgcggcactttgctgcatccccggagctcgagca

SEQ ID NO: 18 - DNA, 4865 bp, Synthetic construct
  source 1..4865: mol_type = other DNA
gcgtataatggactattgtgtgctgatatgtacacttaagtagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagaagcttgccgccaccatgttccccatgcagttcactaatagcgcatacagacaaatggagcccatgtttgctcccggaccaagagggcaggttcaaccttatagacctcggactaagaggcgacaggagcctcaagttggcaatgctgccatcgctgccttggcgaaccagatgtctgctttacagctgcaggtggcagggctagctggtcaggcccgagtcgaccgaagaggacctcggagagtgcagaaatccaagcaaaagaaaaaaaatccatctaatggggagaaacctaaggagaagaagaaaaaacagaagcagcaggaaaagaagggttcaggaggtgttattaaaaagcctcgtaatcggcccgggaaagaggtgcgaattagtgtgaagcgtgcaagacagagcacctttccagtttatcatgacggagcaatttcgggctacgctgtgctgatcgggtcaagggtgttcaaaccagcccacgttaaaggaaaaatcgatcatccggagcttgctgacatcaagtttcaggtcgccgaagacatggacctggaggcagctgcctatcccaagtcgatgcgagatcaagccgctgaacccgcgaccatgatggatggtgtctacaactgggaatatgggacgatccgagtggaggataacgtggttattgacgcatccggccggggcaaaccgggggactctggacgcgccattaccgacaactcaggtaaagtggtgggcatcgtgctcggcggaggtcccgatggccgcaggacccgattatccgttatcggattcgacaagaagatgaaggcaagggagattgcatatagcgaagcaattccctggactcgtgctcctgctctgctgcttcttccaatggtcatcgcatgtacctataactccaacacctttgactgttcaaagcccagttgccaagattgctgtatcaccgcagaaccgaagaaggctatggccatgctgaaagacaatctgaatgacccaaattactgggacctgctcatcgcggtgaccacttgcaactctgctaggaaaaagagagccgtgtccacatctccagcagctgtgtatgacactcagattctggccgcacacgcagcggcctcgccataccgagcttactgccctgattgtgatggtactgcttgcatttctcccattgcaattgatgaggttgtgtcaagcggctctgaccacgtgctccgcattcgtgtgggctcacagtctggagtgactgccaaaggaggtgctgcaggagagacatccttgcgttatctgggccgggatggaaaagtccacgcagcagataatacaaggttggtcgtgcgcaccacagccaaatgtgacgtgttacaagctacaggacactacatcctggcatcctgtcctgaaggacaaagcattacagtggccgctaccctagacggaactcgtcatcagtgcactacagtgtttgaacatcaggtcacagaaaagttcacgagagaaaggtcaaagggtcaccacctgtcagacttaacgaaaaagtgcacccgttttagcaccacaccaaagaaatctgcaccgtatcttgtggatgtttacgacgcgctgccgatctcagtggaaatctccacggtggtcacatgcaatgataaccaatgtaccgtcaaagtgcctccagggaccactgttaagtttgacaaaaaatgtaagtccgctgcacaggcgactgtgactttcactagtgattcccagacattcacatgtgaagaacctgtactcacggctgccagtatcacacaaggaaaacctcatcttcgcagtagcatgttgcccagcggggggaaagaggtgaaagctagaatccccttcccatttccaccagagacggcgacgtgtcgtgtgagtgtggccccactaccaagtataacctacgaggagtctgatgttctcctggccggcacggccaagtaccctgttctcttaaccacaagaaacctgggcttccactctaacgccacttctgagtggattcaaggcaaataccttcgacgcattccagttacccctcaggggatagagctgacctggggcaacaacgcgcctctgcacttttggagttctgttagatatgcctcgggcgatgctgatgcatatccatgggagttgttagtgcatcacacaaaacaccatccagagtacgcctgggcattcgtaggcgttgcctgtggtcttctcgtcatagccgtctgcatgtttgcctgcgcttgtaaccgggtgaggtacagtctggtggccaataccttcaaccctaaccctcctcccctgacagcgctgactgcggcactttgctgcatccccggagctcgagcagatcagccatacttggatataatcgcgtacctgtggactaactcgaaagttgcatttggcctccagtgtgctgctcctgtggcctgtgttctaatagtgacctatgccctgcgccactgccgcttgtgttgcaagagttttcttggcgtaagaggatggtccgcactccttgtgatcctggcgtacgtgcagtcttgtaaaagctacgaacacaccgtggttgttccaatggacccgagagccccatcctatgaggctgtgattaacaggaatggttatgatcctctgaagctgaccatcgcagtcaatttcacagtcatatcaccaactacggctctggagtactggacatgcgctagtgtcccaattgtggagccgccccatgttggctgctgtacatcagttagctgcccgtctgatctatctacactccacgcttttaccggaaaagctgtaagcgatgtgcattgtgacgtacacactaatgtatacccactgctgtggggtgctgcacactgtttctgctccactgaaaacactcaagtctcagctgttgcagcaacagtttccgagttctgtgcccaggacagcgagagagctgaggccttttccgtccattcatctagcgtcacagcggaggttttggtaacgctgggtgaggttgtcactgcagtgcacgtctacgtggatggggtaacgtctgctagaggtacagacctcaagatcgttgccggacccataacaaccgattacagcccgtttgatagaaaggtcgttagaattggtgaggaggtatataactatgactggcctccttacggcgcgggacggccgggcacatttggagatatccaggctcggtcaaccaactacgtaaagccaaacgacctctatggagacataggaattgaagtgttgcagcccaccaatgatcacgtacatgtcgcttacacctacacgacgagtggtctgctacgctggctgcaggatgcccctaaaccgctctctgtcacagccccccatgggtgcaaaattagcgccaatcctcttctggccctggactgcggggtgggagcggtgccgatgagcatcaacatcccagacgcaaaattcacgcgtaaattgaaagatccaaagccttcagccctgaagtgtgtcgtcgacagttgtgagtatggtgttgattatggaggcgcagccactattacatatgagggtcatgaagccgggaagtgtggaatacacagtctcactcctggggtacctctccgcacctcagtcgtggaagttgtagcaggagccaacacagtcaaaactacattctcatcacccacacctgaggtgactcttgaggtcgagatctgctctgccatggtgacctgtgcaagtgaatgcacacccccaaaagaacatgttgtggctactaggcccagacatggctcggacaccggaggatacatcagcggtccagcgatgaggtgggccggcgggattgtcggaacactggctgtgttgttcctgatccttgcagtaacatactgtgtggtgaagaagtgcaggagcaaaaggatcagaatagtgaaatcgtgagaattccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagctagcagatctactagttatcagcacacaattgcccattatacgc

(19) United States
(12) Patent Application Publication
(10) Pub. No.: US 2026/0335424 A1
(43) Pub. Date: Oct. 29, 2026

(54) VACCINE CONSTRUCT AND USES THEREOF

(71) Applicant: CEVA HAMPTON LIMITED, High Wycombe (GB)

(72) Inventors: Ian Thompson, Grange over Sands (GB); Sung Won Kim, Harrow (GB); Lisa Jayne Caproni, Dunblane (GB); Samantha Busfield, Philadelphia, PA (US)

(21) Appl. No.: 19/116,913

(22) PCT Filed: Sep. 29, 2023
(86) PCT No.: PCT/IB2023/059738

§ 371 Date: Mar. 28, 2025

(30) Foreign Application Priority Data
Sep. 29, 2022 (AU) 2022902819

(51) Int. Cl. A61K 39/12; A61K 39/00; A61P 37/04

(52) U.S. Cl. CPC A61K 39/12; A61P 37/04; A61K 2039/53; A61K 2039/54

(57) ABSTRACT

The present disclosure relates generally to vaccine constructs comprising a polynucleotide encoding a salmon alphavirus-like particle and uses thereof, including for inducing an immune response against salmonid alphavirus.

### FIELD OF THE INVENTION

The present disclosure relates generally to a vaccine construct comprising a polynucleotide encoding an alphavirus-like particle and uses thereof.

### BACKGROUND OF THE INVENTION

Salmonid alphavirus (SAV) is an enveloped, single-stranded, positive-sense RNA virus with a ~12 kb genome, belonging to the family Togaviridae, genus Alphavirus. First described in Scotland in 1976, SAV is known to be a serious pathogen for Atlantic salmon (Salmo salar), rainbow trout (Oncorhynchus mykiss) and potentially other salmonid species, where it is responsible for conditions such as pancreas disease (PD). A similar disease, termed “sleeping disease” (SD) and affecting rainbow trout has been described and reported in France. Comparative histopathological and genomic studies suggested both diseases are caused by very similar viruses (Boucher et al., (1996) J Fish Dis 19, 303-310; Weston et al. (2002) J Virol 76, 6155-6163). Recently, SAV has been detected in wild-caught non-salmonid marine fish species, namely flatfish spp. in Europe.

There is some evidence to suggest that infected farmed salmonids are a reservoir and source of contamination of SAV. Both PD and SD are becoming more prevalent, with rising economic importance for aquaculture industries worldwide. Serious quarantine and biosecurity measures have been implemented, with SAV-free countries declining to import salmon products or livestock from regions that have not been declared SAV-free to contain transmission of the pathogen.

Early studies suggested that pre-exposed fish develop some resistance to re-infection Graham et al (2014). Fish. Dis 37:683). Subsequent studies have demonstrated that inactivated virus vaccines, sub-unit protein vaccines and (most recently) DNA vaccines may provide protection against PD (Chang et al. (2017) J. Fish Dis. 40: 1175).

Vaccines against SAV are available, some of which have been reviewed by Deperasińska et al. (J Vet Res. 2018; 62(1): 1-6). Whilst some SAV vaccines have proven to be modestly effective at reducing mortality, most have generally failed to prevent pathological lesions in the pancreas and heart, or growth retardation. However, despite the introduction of these vaccines and increased biosecurity measures, SAV continues to be a major health and welfare issue in salmonid aquaculture, with 100 new cases registered in the most affected country (Norway) in 2021, and 98 new cases in 2022 (Norwegian Veterinary Institute, Norwegian Fish Health Report 2022 Norwegian Fish Health Report 222 (vetinst.no).

Therefore, there remains an urgent need for an effective vaccine for inducing protective immunity against SAV infection.

### SUMMARY OF THE INVENTION

In an aspect disclosed herein, there is provided vaccine construct comprising a polynucleotide encoding a salmon alphavirus-like particle, wherein the polynucleotide comprises a nucleic acid sequence encoding a salmon alphavirus capsid protein and at least one nucleic acid sequence encoding a salmon alphavirus envelope protein selected from the group consisting of an E3, E2, 6K and E1 envelope protein, wherein the nucleic acid sequence encoding the salmon alphavirus capsid protein and/or the at least one nucleic acid sequence encoding the salmon alphavirus envelope protein is codon-optimised to resemble codon usage in a fish cell, and wherein the polynucleotide provides an enhanced immune response against salmon alphavirus when compared to a polynucleotide encoding a salmon alphavirus-like particle in which the nucleic acid sequences are not codon-optimised.

In an aspect disclosed herein, there is provided a vaccine construct comprising polynucleotide encoding a salmon alphavirus-like particle, wherein the polynucleotide comprises:
(a) a nucleic acid sequence encoding a salmon alphavirus capsid protein, wherein the nucleic acid sequence encoding the salmon alphavirus capsid protein comprises the nucleic acid sequence set forth in SEQ ID NO:5 or a nucleic acid sequence having at least 76.5% sequence identity thereto; and
(b) at least one nucleic acid sequence encoding a salmon alphavirus envelope protein selected from the group consisting of an E3, E2, 6K and E1 envelope protein, wherein:
   (i) the nucleic acid sequence encoding the E3 envelope protein comprises the nucleic acid sequence set forth in SEQ ID NO:7 or a nucleic acid sequence having at least 80.5% sequence identity thereto;
   (ii) the nucleic acid sequence encoding the E2 envelope protein comprises the nucleic acid sequence set forth in SEQ ID NO:9 or a nucleic acid sequence having at least 74.5% sequence identity thereto;
   (iii) the nucleic acid sequence encoding the 6K envelope protein comprises the nucleic acid sequence set forth in SEQ ID NO:11 or a nucleic acid sequence having at least 73% sequence identity thereto; and
   (iv) the nucleic acid sequence encoding the E1 envelope protein comprises the nucleic acid sequence set forth in SEQ ID NO:13 or a nucleic acid sequence having at least 76.5% sequence identity thereto.

In an embodiment, (a) the nucleic acid sequence encoding the salmon alphavirus capsid protein comprises the nucleic acid sequence set forth in SEQ ID NO:5; (b) the nucleic acid sequence encoding the E3 envelope protein comprises the nucleic acid sequence set forth in SEQ ID NO:7; (c) the nucleic acid sequence encoding the E2 envelope protein comprises the nucleic acid sequence set forth in SEQ ID NO:9; (d) the nucleic acid sequence encoding the 6K envelope protein comprises the nucleic acid sequence set forth in SEQ ID NO:11; and (e) the nucleic acid sequence encoding the E1 envelope protein comprises the nucleic acid sequence set forth in SEQ ID NO:13.

In an embodiment, (a) the nucleic acid sequence encoding the salmon alphavirus capsid protein consists of the nucleic acid sequence set forth in SEQ ID NO:5; (b) the nucleic acid sequence encoding the E3 envelope protein consists of the nucleic acid sequence set forth in SEQ ID NO:7; (c) the nucleic acid sequence encoding the E2 envelope protein consists of the nucleic acid sequence set forth in SEQ ID NO:9; (d) the nucleic acid sequence encoding the 6K envelope protein consists of the nucleic acid sequence set forth in SEQ ID NO:11; and (e) the nucleic acid sequence encoding the E1 envelope protein consists of the nucleic acid sequence set forth in SEQ ID NO:13.

In an embodiment, the polynucleotide comprises the nucleic acid sequence set forth in SEQ ID NO:3 or a nucleic acid sequence having at least 76% sequence identity thereto.

In an embodiment, the polynucleotide consists of the nucleic acid sequence set forth in SEQ ID NO:3.

In an embodiment, the nucleic acid sequence encoding the salmon alphavirus capsid protein is operatively linked to a promoter.

In an embodiment, the promoter is a CMV promoter or an EF1a promoter.

In an embodiment, the at least one nucleic acid sequence encoding the salmon alphavirus envelope protein is operatively linked to a promoter.

In an embodiment, the promoter is a CMV promoter or an EFla promoter.

In an embodiment, the polynucleotide further comprises a SV40 poly(A) sequence.

In an embodiment, the polynucleotide further comprising a telomeric sequence.

In an embodiment, telomeric sequences flank both ends of the polynucleotide construct.

In an embodiment, the polynucleotide is a closed linear polynucleotide.

The present disclosure also extends to a cell comprising the polynucleotide described herein.

The present disclosure also extends to a salmon alphavirus-like particle encoded by the polynucleotide described herein.

The present disclosure also extends to a vaccine composition comprising the vaccine construct, the cell or the salmon alphavirus-like particle described herein.

The present disclosure also extends to a method for treating or protecting against salmon alphavirus infection in a subject, the method comprising administering to a subject in need thereof the vaccine construct, the salmon alphavirus-like particle or the composition described herein.

In an embodiment, the vaccine construct, the salmon alphavirus-like particle or the composition described herein is administered to the subject by intramuscular injection.

In an embodiment, the method described herein comprises administering to the subject the vaccine construct in an amount of from about 0.5 micrograms to about 10 micrograms.

In an embodiment, the subject has, or is at risk of developing, salmonoid pancreatic disease or sleeping disease.

The present disclosure also extends to a use of the vaccine construct, the cell or the salmon alphavirus-like particle described herein, in the manufacture of a vaccine composition for treating or protecting against salmon alphavirus infection in a subject.

The present disclosure also extends to the vaccine construct described herein for use in treating or preventing against salmon alphavirus infection in a subject.

The present disclosure also extends to a method of inducing an immune response against salmonid alphavirus in a subject, the method comprising administering to a subject in need thereof the vaccine construct, the salmon alphavirus-like particle or the composition described herein.

In an embodiment, the salmonid alphavirus is a SAV2 subtype.

In an embodiment, the salmonid alphavirus is a SAV3 subtype.

In an embodiment, the immune response comprises an antibody response, wherein the antibodies are capable of binding specifically to and neutralising the salmon alphavirus.

In an embodiment, the antibodies are capable of binding specifically to and neutralising SAV2 and SAV3 subtypes.

In an embodiment, the polynucleotide, the salmon alphavirus-like particle or the composition described herein is administered to the subject by intramuscular injection.

### BRIEF DESCRIPTION OF THE DRAWINGS

Embodiments of the disclosure are described herein, by way of non-limiting example only, with reference to the following figures:

FIG. 1 shows a schematic of the cohabitation challenge model trial design. DD—Degree days; a means of standardising treatment times in studies with poikilothermic animals (for example, 10 days exposure at 10 °C=100 degree days). DPC=days post challenge.

FIG. 2 shows a schematic of Doggybone closed linear constructs (dbDNA) comprising a polynucleotide construct comprising capsid, E3, E2, 6K and E1 nucleic acid sequences encoding for a salmon alphavirus-like particle (A). The nucleotide and corresponding amino acid sequences of the Doggybone closed linear constructs (dbDNA) comprising a polynucleotide construct comprising capsid, E3, E2, 6K and E1 nucleic acid sequences encoding for a salmon alphavirus-like particle (B; see also SEQ ID NO:14).

FIG. 3 shows a schematic of Doggybone closed linear constructs (dbDNA) comprising a polynucleotide construct comprising codon-optimised modified capsid, E3, E2, 6K and E1 nucleic acid sequences encoding for a salmon alphavirus-like particle (A). The codon optimised nucleotide and corresponding amino acid sequences of the Doggybone closed linear constructs (dbDNA) comprising a polynucleotide construct comprising capsid, E3, E2, 6K and E1 nucleic acid sequences encoding for a salmon alphavirus-like particle (B; see also SEQ ID NO:15).

FIG. 4 shows SAV viraemia post-challenge expressed as SAV genome copy number (log₁₀; mean+S.E.M.) in fish receiving negative control (-ve Control; dbDNA eGFP 7.5 μg), 3-dbDNA SAV3 (7.5 μg); or ClyNav® at the recommended dose of 6.0-9.4 μg plasmid DNA per 0.05 ml.

FIG. 5 shows SAV viraemia post-challenge expressed as SAV genome copy number (log₁₀; mean+S.E.M.) in fish receiving negative control dbDNA eGFP (-ve Control), very low dose of 3-dbDNA SAV3 (1.0 μg); or very low dose of 5-dbDNA SAV3 CO (1.0 μg).

FIG. 6 shows SAV3 neutralising antibody titres in fish receiving a reference dose (7.5 μg) of either 3-dbDNA SAV3 or 5-dbDNA SAV3 CO, at 500 degree days post-immunisation, with individual data points around the median.

FIG. 7 shows SAV3 neutralising antibody titres in fish receiving a reference dose of 5-dbDNA SAV3 CO (7.5 μg) or recommended dose of ClyNav®, at 500 degree days post-immunisation, with individual data points around the median.

FIG. 8 shows SAV2 and SAV3 neutralising antibody titres in fish receiving a reference dose of 5-dbDNA SAV3 CO (7.5 μg) or recommended dose of ClyNav®, at 500 degree days post-immunisation, with individual data points around the median.

FIG. 9 shows the nucleotide and polypeptide sequences disclosed herein.

### DETAILED DESCRIPTION OF THE INVENTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described.

Nucleotide and amino acid sequences are referred to by sequence identifier numbers (SEQ ID NO:). The SEQ ID NOs: correspond numerically to the sequence identifiers <400>1, <400>2, etc. A summary of sequence identifiers is provided herein.

All sequence reference numbers (e.g., GenBank ID, EMBL-Bank ID, DNA Data Bank of Japan (DDBK) ID, etc.) provided herein were current as at the filing date.

The present disclosure is predicated, at least in part, on the inventors' surprising finding that a vaccine construct encoding a salmon alphavirus-like particle, wherein the polynucleotide comprises nucleic acid sequences that have been codon-optimised to resemble codon usage in a fish cell, unexpectedly provides improved protection against salmon alphavirus infection when compared to a polynucleotide encoding a salmon alphavirus-like particle in which the nucleic acid sequences are not codon-optimised. This finding is entirely surprising, as one would have expected salmon alpha virus to have naturally evolved towards optimal codon usage in fish cells.

For the purposes of the present invention, the following terms are defined below.

The articles “a” and “an” are used herein to refer to one or to more than one (i.e., to at least one) of the grammatical object of the article. By way of example, “an element” means one element or more than one element.

As used herein, “and/or” refers to and encompasses any and all possible combinations of one or more of the associated listed items, as well as the lack of combinations when interpreted in the alternative (or).

The terms “active agent” and “therapeutic agent” are used interchangeably herein and refer to agents that prevent, reduce or ameliorate at least one symptom of a disease or disorder.

The terms “administration concurrently” or “administering concurrently” or “co-administering” and the like refer to the administration of a single composition containing two or more agents, or the administration of each agent as separate compositions and/or delivered by separate routes either contemporaneously or simultaneously or sequentially within a short enough period of time that the effective result is equivalent to that obtained when all such agents are administered as a single composition. By “simultaneously” is meant that the agents are administered at substantially the same time, and desirably together in the same formulation. By “contemporaneously” it is meant that the agents are administered closely in time, e.g., one agent is administered within from about one minute to within about one day before or after another. Any contemporaneous time is useful. However, it will often be the case that when not administered simultaneously, the agents will be administered within about one minute to within about eight hours and suitably within less than about one to about four hours. When administered contemporaneously, the agents are suitably administered at the same site on the subject. The term “same site” includes the exact location, but can be within about 0.5 to about 15 centimeters, preferably from within about 0.5 to about 5 centimeters. The term “separately” as used herein means that the agents are administered at an interval, for example at an interval of about a day to several weeks or months. The agents may be administered in either order. The term “sequentially” as used herein means that the agents are administered in sequence, for example at an interval or intervals of minutes, hours, days or weeks. If appropriate the agents may be administered in a regular repeating cycle.

Throughout this specification, unless the context requires otherwise, the words “comprise”, “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The term “corresponding” as used herein in reference to a particular gene is intended to mean an analogous or equivalent or comparable gene. For example, where reference is made to a corresponding endogenous gene, it is intended to mean the analogous, equivalent or comparable naturally-occurring gene. Where reference is made to a corresponding exogenous gene, it is intended to mean an analogous, equivalent or comparable exogenous gene. In some embodiments, the corresponding gene has analogous or equivalent function or having sequence similarity. In one embodiment, the corresponding gene may be identical in function and/or sequence. In another embodiment, the corresponding gene may have about the same function or activity. In another embodiment, the corresponding gene may have reduced function or activity. In some embodiments, the phrase “corresponds to” or “corresponding to” is meant a nucleic acid sequence that displays substantial sequence identity to a reference nucleic acid sequence. In general the nucleic acid sequence will display at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or even up to 100% sequence identity to the reference nucleic acid sequence.

As used herein, the terms “encode,” “encoding” and the like refer to the capacity of a nucleic acid to provide for another nucleic acid or a polypeptide. For example, a nucleic acid sequence is said to “encode” a polypeptide if it can be transcribed and/or translated, typically in a host cell, to produce the polypeptide or if it can be processed into a form that can be transcribed and/or translated to produce the polypeptide. Such a nucleic acid sequence may include a coding sequence or both a coding sequence and a non-coding sequence. Thus, the terms “encode,” “encoding” and the like include an RNA product resulting from transcription of a DNA molecule, a protein resulting from translation of an RNA molecule, a protein resulting from transcription of a DNA molecule to form an RNA product and the subsequent translation of the RNA product, or a protein resulting from transcription of a DNA molecule to provide an RNA product, processing of the RNA product to provide a processed RNA product (e.g., mRNA) and the subsequent translation of the processed RNA product.

By “effective amount”, in the context of treating a disease or condition is meant the administration of an amount of an agent or composition to an individual in need of such treatment or prophylaxis, either in a single dose or as part of a series, that is effective for the prevention of incurring a symptom, holding in check such symptoms, and/or treating existing symptoms, of that condition. The effective amount will vary depending upon the age, health and physical condition of the individual to be treated and whether symptoms of disease are apparent, the taxonomic group of individual to be treated, the formulation of the composition, the assessment of the medical situation, and other relevant factors. Optimal dosing schedules can be calculated from measurements of drug accumulation in the body of the subject. Optimum dosages may vary depending on the relative potency in an individual subject, and can generally be estimated based on EC50 values found to be effective in in vitro and in vivo animal models. Persons of ordinary skill can easily determine optimum dosages, dosing methodologies and repetition rates. It is expected that the amount will fall in a relatively broad range that can be determined through routine trials.

The term “expression”, as used herein, typically refers to any step involved in the production of an RNA molecule or a polypeptide, such as by transcription, post-transcriptional modification, translation, post-translational modification, and secretion.

By “isolated” is meant material that is substantially or essentially free from components that normally accompany it in its native state.

As used herein, the term “nucleic acid”, “nucleic sequence”, “polynucleotide”, “oligonucleotide” and “nucleotide sequence” as used herein refers to mRNA, RNA, cRNA, rRNA, cDNA, or DNA, or a combination thereof. The term typically refers to polymeric form of nucleotides, either ribonucleotides or deoxynucleotides or a modified form of either type of nucleotide. The term includes single-, double- or triple-stranded forms of DNA and RNA. It can be of recombinant, artificial and/or synthetic origin and it can comprise modified nucleotides, comprising for example a modified bond, a modified purine or pyrimidine base, or a modified sugar. The nucleic acids of the present disclosure can be in isolated or purified form, and made, isolated and/or manipulated by techniques known per se in the art, e.g., cloning and expression of cDNA libraries, amplification, enzymatic synthesis or recombinant technology. The nucleic acids can also be synthesised in vitro by well-known chemical synthesis techniques, as described in, e.g., Belousov (1997) Nucleic Acids Res. 25:3440-3444.

The terms “peptide”, “polypeptide” and “protein” are to be understood as referring to a chain of amino acids linked by peptide bonds, irrespective of the number of amino acids forming said chain. Amino acids are typically represented by their one-letter or three-letters code, according to the following nomenclature: A: alanine (Ala); C: cysteine (Cys); D: aspartic acid (Asp); E: glutamic acid (Glu); F: phenylalanine (Phe); G: glycine (Gly); H: histidine (His); I: isoleucine (Ile); K: lysine (Lys); L: leucine (Leu); M: methionine (Met); N: asparagine (Asn); P: proline (Pro); Q: glutamine (Gln); R: arginine (Arg); S: serine (Ser); T: threonine (Thr); V: valine (Val); W: tryptophan (Trp) and Y: tyrosine (Tyr).

A “promoter” refers to one or more a nucleic acid control sequences that direct transcription of a nucleic acid. As used herein, a promoter may include necessary nucleic acid sequences near the start site of transcription, such as, in the case of a polymerase II type promoter, a TATA element. A promoter may optionally include distal enhancer or repressor elements, which can be located as much as several thousand base pairs from the start site of transcription. “Promoter” includes a minimal promoter that is a short nucleic acid sequence comprised of a TATA-box and other sequences that serve to specify the site of transcription initiation, to which control elements (e.g., cis-acting elements) are added for control of expression. “Promoter” also refers to a nucleotide sequence that includes a minimal promoter plus control elements (e.g., cis-acting elements) that are capable of controlling the expression of a coding sequence or functional RNA. This type of promoter sequence consists of proximal and more distal upstream elements, the latter elements often referred to as enhancers. Accordingly, an “enhancer” is a nucleic acid sequence which can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific nucleic acid-binding proteins that mediate their effects. Promoters may be derived in their entirety from a native gene, or be composed of different elements derived from different promoters found in nature, or even be comprised of synthetic nucleic acid segments. A promoter may also contain nucleic acid sequences that are involved in the binding of protein factors which control the effectiveness of transcription initiation in response to physiological or developmental conditions. Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

The term “sequence identity” as used herein refers to the extent that sequences are identical on a nucleotide-by-nucleotide basis or an amino acid-by-amino acid basis over a window of comparison (e.g., over 10, 15, 20, 30, 40, 50, 60, 70, 80, 90, 100, 120, 140, 160, 180, 200 or more nucleotides or amino acids residues). Thus, a “percentage of sequence identity” is calculated by comparing two optimally aligned sequences over the window of comparison, determining the number of positions at which the identical nucleic acid base (e.g., A, T, C, G) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present disclosure, “sequence identity” will be understood to mean the “match percentage” calculated by an appropriate method. For example, sequence identity analysis may be carried out using the DNASIS computer program (Version 2.5 for windows; available from Hitachi Software engineering Co., Ltd., South San Francisco, California, USA) using standard defaults as used in the reference manual accompanying the software. Sequences may be aligned using a global alignment algorithms (e.g., Needleman and Wunsch algorithm; Needleman and Wunsch, 1970), which aligns the sequences optimally over the entire length, while sequences of substantially different lengths are preferably aligned using a local alignment algorithm (e.g., Smith and Waterman algorithm (Smith and Waterman, 1981) or Altschul algorithm (Altschul et al., 1997; Altschul et al., 2005)). Alignment for the purposes of determining percent amino acid sequence identity can be achieved by any means available to persons skilled in the art, illustrative examples of which include publicly available computer software, such as is available at http://blast.ncbi.nlm.nih.gov/ or http://www.ebi.ac.uk/Tools/emboss/). Persons skilled in the art can readily determine appropriate parameters for measuring alignment, including any algorithms needed to achieve maximal alignment over the full length of the sequences being compared. As used herein, % sequence identity typically refers to values generated using pair wise sequence alignment that creates an optimal global alignment of two sequences (e.g., using the Needleman-Wunsch algorithm).

The term “sequence identity”, as used herein, includes exact identity between compared sequences at the nucleotide or amino acid level. Sequence identity, as herein described, typically relates to the percentage of amino acid residues in the candidate sequence that are identical with the residues of the corresponding peptide sequence after aligning the sequences and introducing gaps, if necessary, to achieve the maximum percentage homology, and not considering any conservative substitutions as part of the sequence identity. Neither N- or C-terminal extensions, nor insertions shall be construed as reducing sequence identity or homology.

The present disclosure also extends to non-exact identity (i.e., similarity) of sequences at the nucleotide or amino acid level where any difference(s) between sequences are in relation to amino acids (or in the context of nucleotides, amino acids encoded by said nucleotides) that are nevertheless related to each other at the structural, functional, biochemical and/or conformational levels. For example, where there is non-identity (similarity) at the amino acid level, “similarity” includes amino acids that are nevertheless related to each other at the structural, functional, biochemical and/or conformational levels. In an embodiment, nucleotide and sequence comparisons are made at the level of identity rather than similarity. For example, leucine may be substituted for an isoleucine or valine residue. This may be referred to as a conservative substitution. In an embodiment, the amino acid sequences may be modified by way of conservative substitution of any of the amino acid residues contained therein, such that the modification has no or negligible effect on the functional activity of the modified polypeptide when compared to the unmodified polypeptide.

Each embodiment described herein is to be applied mutatis mutandis to each and every embodiment unless specifically stated otherwise.

### Salmon Alphavirus

As noted elsewhere herein, salmonid alphavirus (SAV) is an enveloped, single-stranded, positive-sense RNA virus with a ~12 kb genome, belonging to the family Togaviridae, genus Alphavirus. The genomes of the reference strains of the viruses thought to cause PD and SD have been sequenced and compared demonstrating that these strains are subtypes of the same virus, as suggested by an earlier comparative histopathology study. Hereafter, the terms SAV and SPDV are used interchangeably, and understood to mean salmonid alphaviruses that underlie PD and SD in salmonid species. Based on genomic analyses, at least six subtypes of SAV have been described that are the causative agents of significant diseases of farmed Atlantic salmon, rainbow trout and other salmonids (SAV1-6). In Europe, SAV1 causes PD in farmed Atlantic salmon in Ireland. SAV2 or SDV causes sleeping disease in England, France, Germany, Italy, Scotland and Spain. SAV3 or Norwegian salmon alphavirus is the original subtype identified in Norway. SAV4 consists of Atlantic salmon strains from Ireland, SAV5 consists of Scottish strains and SAV6 contains one virus, isolated from Atlantic salmon in Ireland. SAV2 has been divided into two subgroups named freshwater variant (SAV2 FW) and marine variant (SAV2 MW). Infections with SAV2 FW cause sleeping disease in freshwater-reared rainbow trout in England, Scotland, and mainland European countries. The marine variant of SAV2 (SAV2 MW) is responsible for pancreas disease in seawater-reared Atlantic salmon. The third subtype of salmonid alphavirus (SAV3) has been isolated from farmed Atlantic salmon in Norway, hence it is also named Norwegian salmonid alphavirus (NSAV). SAV3 causes pancreas disease in Atlantic salmon and sea-reared rainbow trout. It has been shown that SAV3 has genomic organisation identical to that of SAV1 and SAV2. There are currently two PD epidemics ongoing in Norway. SAV3 was the only subtype recognised in Norway until 2010, but since that time, SAV2 has become well established. Subtypes 4-6 of SAV (SAV4-6) have been detected in Scotland and Ireland in connection with PD outbreaks and detected there along with SAV1.

The transmission vector for SAV has not been identified, but direct horizontal transmission has been demonstrated. It is not known whether aquatic invertebrates play a significant role in the epizootiology of the SAV-associated diseases. The main transmission route of salmonid alphavirus appears to be horizontal and via water contact, while there is suggestion that vertical transfer (parent to offspring) may be possible as well. All life stages are considered susceptible to infection with SAV. Transport of fish across borders or different waterways will likely increase the risk of spreading SAV (reviewed by Jansen et al. (2017) Journal of Fish Diseases 40(1):141).

Clinical signs associated with PD include sudden inappetence, lethargy and an increased number of faecal casts in the cages, increased mortality and ill-thrift. The histopathological changes in fish affected by PD and SD primarily occur in the pancreas, heart, and skeletal muscles. Of the six different SAV subtypes, SAV1 and SAV3 are associated with cardiovascular and muscular tissue pathologies (Graham et al. (2011) J Fish Dis 34:273). Changes in the dermal bacterial microflora composition of Atlantic salmon (characterised most prominently by the loss of Proteobacteria) has also been detected in response to infection with SAV3 which may render the fish more susceptible to secondary infections by opportunistic bacterial pathogens present in the environment or within the host indigenous microbial reservoir.

Sleeping disease (SD) is an infectious disease similar to pancreas disease; however, it affects rainbow trout reared in fresh water. SD is an increasing problem throughout Europe, causing high mortality and growth retardation of fish. The characteristic sign of sleeping disease is the unusual behaviour of affected fish, manifested in their laying on their side on the bottom of the tank, hence the name “sleeping” disease. Extensive necrosis of skeletal red muscles is considered to be the cause of this behaviour. This is followed by the characteristic development of histological lesions of the pancreas and heart.

Based on the field observations showing that fish surviving SAV infections were less susceptible to reinfection, it has been suggested that vaccination may be applied for control of PD and SD, although efficacious vaccines remain elusive.

### Polynucleotides

The present disclosure provides a vaccine construct comprising polynucleotide encoding a salmon alphavirus-like particle, wherein the polynucleotide comprises a nucleic acid sequence encoding a salmon alphavirus capsid protein and at least one nucleic acid sequence encoding a salmon alphavirus envelope protein selected from the group consisting of an E3, E2, 6K and E1 envelope protein, wherein the nucleic acid sequence encoding the salmon alphavirus capsid protein and/or the at least one nucleic acid sequence encoding the salmon alphavirus envelope protein is codon-optimised to resemble codon usage in a fish cell, and wherein the polynucleotide provides an enhanced immune response against salmon alphavirus when compared to a polynucleotide encoding a salmon alphavirus-like particle in which the nucleic acid sequences are not codon-optimised.

The present disclosure also provides a vaccine construct comprising a polynucleotide encoding a salmon alphavirus-like particle, wherein the polynucleotide comprises:
(a) a nucleic acid sequence encoding a salmon alphavirus capsid protein, wherein the nucleic acid sequence encoding the salmon alphavirus capsid protein comprises the nucleic acid sequence set forth in SEQ ID NO:5 or a nucleic acid sequence having at least 76.5% sequence identity thereto; and
(b) at least one nucleic acid sequence encoding a salmon alphavirus envelope protein selected from the group consisting of an E3, E2, 6K and E1 envelope protein, wherein:
   (i) the nucleic acid sequence encoding the E3 envelope protein comprises the nucleic acid sequence set forth in SEQ ID NO:7 or a nucleic acid sequence having at least 80.5% sequence identity thereto;
   (ii) the nucleic acid sequence encoding the E2 envelope protein comprises the nucleic acid sequence set forth in SEQ ID NO:9 or a nucleic acid sequence having at least 74.5% sequence identity thereto;
   (iii) the nucleic acid sequence encoding the 6K envelope protein comprises the nucleic acid sequence set forth in SEQ ID NO:11 or a nucleic acid sequence having at least 73% sequence identity thereto; and
   (iv) the nucleic acid sequence encoding the E1 envelope protein comprises the nucleic acid sequence set forth in SEQ ID NO:13 or a nucleic acid sequence having at least 76.5% sequence identity thereto.

Virus-like particles (VLP) have been shown to be useful as vaccines against a variety of infectious agents, including viral and bacterial infections. VLP are self-assembling complexes of capsid and/or envelope proteins (also referred to herein as viral structural proteins) that mimic the overall structure of their parental virus. VLP may also lack or possess dysfunctional copies of certain genes of the native virus, and this may result in the virus-like-particle being incapable of some function that is otherwise characteristic of the native virus, such as replication and/or cell-cell movement. Typically void of viral genetic material, VLP possess biologically desirable traits that are attributed, at least in part, to the particulate viral structure. Of particular interest is their efficient recognition, cellular uptake, and processing by host immune systems. VLP are also amenable to a broad range of modifications including encapsulation, chemical conjugation, and genetic manipulation (see, e.g., Roldio et al. Expert Rev Vaccines 2010; 9(10):1149-76), allowing VLP to be employed as suitable delivery agents for immunotherapy, illustrative examples of which include the prophylactic VLP vaccines Gardasil®, Cervarix®, Hecolin®, and Porcilis PCV®. VLP also overcome some of the drawbacks associated with traditional vaccine production; namely, the infectious nature associated with live and inactivated vaccines and lengthy production time.

The term “self-assembly” typically refers to a process in which a system of pre-existing components, under specific conditions, adopts a more organised structure through interactions between the components themselves. In the present context, self-assembly refers to the intrinsic capacity of the encoded structural proteins (the capsid and the one or more envelope proteins), once expressed, to self-assemble into VLP, including in the absence of other viral proteins. “Self-assembly” does not preclude the possibility that cellular proteins such as chaperones participate in the process of intracellular VLP assembly. The self-assembly process may be influenced by factors such as choice of expression host, choice of expression conditions, and conditions for maturing the VLP. Virus capsid and/or envelope proteins may be able to form VLP on their own, or in combination with several virus capsid and/or envelope proteins, these optionally all being identical or related essential components of the virus structure.

The terms “virus-like particle” and “VLP” are therefore used interchangeably herein to refer to one or several recombinantly expressed viral structural (capsid and envelope) proteins, which spontaneously assemble into macromolecular particulate structures mimicking the morphology of a virus coat, but lacking infectious genetic material. As noted elsewhere herein, the polynucleotide encodes a salmon alphavirus and at least one (e.g., 1, 2, 3 or 4) salmon alphavirus envelope proteins, preferably at least three or more preferably at least four salmon alphavirus envelope proteins. In an embodiment, the polynucleotide encodes a salmon alphavirus VLP comprising, consisting or consisting essentially of a salmon alphavirus capsid protein and one salmon alphavirus envelope protein. In an embodiment, the polynucleotide encodes a salmon alphavirus VLP comprising, consisting or consisting essentially of a salmon alphavirus capsid protein and two salmon alphavirus envelope proteins. In an embodiment, the polynucleotide encodes a salmon alphavirus VLP comprising, consisting or consisting essentially of a salmon alphavirus capsid protein and three salmon alphavirus envelope proteins. In an embodiment, the polynucleotide encodes a salmon alphavirus VLP comprising, consisting or consisting essentially of a salmon alphavirus capsid protein and four salmon alphavirus envelope proteins. It is to be understood that the VLP may comprise any number of two or more viral structural proteins, including any combination of viral capsid and/or envelope proteins, as long as the two or more viral structural proteins, once liberated following host cell expression, are capable of self-assembly to form a VLP.

In an embodiment, the nucleic acid sequence encoding the salmon alphavirus capsid protein comprises, consists or consists essentially of the nucleic acid sequence of SEQ ID NO:5. In another embodiment, the nucleic acid sequence encoding the salmon alphavirus capsid protein comprises, consists or consists essentially of the nucleic acid sequence having at least 76.5% sequence identity to SEQ ID NO:5. By “at least 76.5% sequence identity” is meant 76.5%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO:5, for example, after optimal alignment or best fit analysis. Thus, in an embodiment, the nucleic acid sequence encoding the salmon alphavirus capsid protein comprises, consists or consists essentially of a nucleic acid sequence having at least 76.5%, at least 77%, preferably at least 78%, preferably at least 79%, preferably at least 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98% or more preferably at least 99% sequence identity to SEQ ID NO:5, for example, after optimal alignment or best fit analysis. It is to be understood that a salmon alphavirus capsid protein encoded by a nucleic acid sequence having at least 76.5% sequence identity to SEQ ID NO:5, as herein described, will suitably be a “functional variant” of the reference sequence (i.e., of SEQ ID NO:5). It is to be understood that a “functional variant”, as used herein, means a nucleotide sequence that differs from the reference sequence to which it is being compared, which may include a natural (i.e., native) sequence or a synthetic variant thereof, yet encodes a salmon alphavirus capsid protein capable of self-assembly (with other viral structural proteins) to form a VLP, as described herein.

In an embodiment, the nucleic acid sequence encoding the salmon alphavirus E3 envelope protein comprises, consists or consists essentially of the nucleic acid sequence of SEQ ID NO:7. In another embodiment, the nucleic acid sequence encoding the salmon alphavirus E3 envelope protein comprises, consists or consists essentially of the nucleic acid sequence having at least 80.5% sequence identity to SEQ ID NO:7. By “at least 80.5% sequence identity” is meant 80.5%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO:7, for example, after optimal alignment or best fit analysis. Thus, in an embodiment, the nucleic acid sequence encoding the salmon alphavirus E3 envelope protein comprises, consists or consists essentially of a nucleic acid sequence having at least 80.5%, having at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98% or more preferably at least 99% sequence identity to SEQ ID NO:7, for example, after optimal alignment or best fit analysis. It is to be understood that a salmon alphavirus E3 envelope protein encoded by a nucleic acid sequence having at least 80.5% sequence identity to SEQ ID NO:7, as herein described, will suitably be a “functional variant” of the reference sequence (i.e., of SEQ ID NO:7). It is to be understood that a “functional variant”, as used herein, means a nucleotide sequence that differs from the reference sequence to which it is being compared, which may include a natural (i.e., native) sequence or a synthetic variant thereof, yet encodes a salmon alphavirus E3 envelope protein capable of self-assembly (with other viral structural proteins) to form a VLP, as described herein.

In an embodiment, the nucleic acid sequence encoding the salmon alphavirus E2 envelope protein comprises, consists or consists essentially of the nucleic acid sequence of SEQ ID NO:9. In another embodiment, the nucleic acid sequence encoding the salmon alphavirus E2 envelope protein comprises, consists or consists essentially of the nucleic acid sequence having at least 74.5% sequence identity to SEQ ID NO:9. By “at least 74.5% sequence identity” is meant 74.5%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO:9, for example, after optimal alignment or best fit analysis. Thus, in an embodiment, the nucleic acid sequence encoding the salmon alphavirus E2 envelope protein comprises, consists or consists essentially of a nucleic acid sequence having at least 74.5%, having at least 75%, preferably at least 76%, preferably at least 77%, preferably at least 78%, preferably at least 79%, preferably at least 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98% or more preferably at least 99% sequence identity to SEQ ID NO:9, for example, after optimal alignment or best fit analysis. It is to be understood that a salmon alphavirus E2 envelope protein encoded by a nucleic acid sequence having at least 74.5% sequence identity to SEQ ID NO:9, as herein described, will suitably be a “functional variant” of the reference sequence (i.e., of SEQ ID NO:9). It is to be understood that a “functional variant”, as used herein, means a nucleotide sequence that differs from the reference sequence to which it is being compared, which may include a natural (i.e., native) sequence or a synthetic variant thereof, yet encodes a salmon alphavirus E2 envelope protein capable of self-assembly (with other viral structural proteins) to form a VLP, as described herein.

In an embodiment, the nucleic acid sequence encoding the salmon alphavirus 6K envelope protein comprises, consists or consists essentially of the nucleic acid sequence of SEQ ID NO:11. In another embodiment, the nucleic acid sequence encoding the salmon alphavirus 6K envelope protein comprises, consists or consists essentially of the nucleic acid sequence having at least 73% sequence identity to SEQ ID NO:11. By “at least 73% sequence identity” is meant 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO:11, for example, after optimal alignment or best fit analysis. Thus, in an embodiment, the nucleic acid sequence encoding the salmon alphavirus 6K envelope protein comprises, consists or consists essentially of a nucleic acid sequence having at least 73%, having at least 74%, preferably at least 75%, preferably at least 76%, preferably at least 77%, preferably at least 78%, preferably at least 79%, preferably at least 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98% or more preferably at least 99% sequence identity to SEQ ID NO:11, for example, after optimal alignment or best fit analysis. It is to be understood that a salmon alphavirus 6K envelope protein encoded by a nucleic acid sequence having at least 73% sequence identity to SEQ ID NO:11, as herein described, will suitably be a “functional variant” of the reference sequence (i.e., of SEQ ID NO:11). It is to be understood that a “functional variant”, as used herein, means a nucleotide sequence that differs from the reference sequence to which it is being compared, which may include a natural (i.e., native) sequence or a synthetic variant thereof, yet encodes a salmon alphavirus 6K envelope protein capable of self-assembly (with other viral structural proteins) to form a VLP, as described herein.

In an embodiment, the nucleic acid sequence encoding the salmon alphavirus E1 envelope protein comprises, consists or consists essentially of the nucleic acid sequence of SEQ ID NO:13. In another embodiment, the nucleic acid sequence encoding the salmon alphavirus E1 envelope protein comprises, consists or consists essentially of the nucleic acid sequence having at least 76.5% sequence identity to SEQ ID NO:13. By “at least 76.5% sequence identity” is meant 76.5%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO:13, for example, after optimal alignment or best fit analysis. Thus, in an embodiment, the nucleic acid sequence encoding the salmon alphavirus E1 envelope protein comprises, consists or consists essentially of a nucleic acid sequence having at least 76.5%, 77%, 78%, preferably at least 79%, preferably at least 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98% or more preferably at least 99% sequence identity to SEQ ID NO:13, for example, after optimal alignment or best fit analysis. It is to be understood that a salmon alphavirus E1 envelope protein encoded by a nucleic acid sequence having at least 76.5% sequence identity to SEQ ID NO:13, as herein described, will suitably be a “functional variant” of the reference sequence (i.e., of SEQ ID NO:13). It is to be understood that a “functional variant”, as used herein, means a nucleotide sequence that differs from the reference sequence to which it is being compared, which may include a natural (i.e., native) sequence or a synthetic variant thereof, yet encodes a salmon alphavirus E1 envelope protein capable of self-assembly (with other viral structural proteins) to form a VLP, as described herein.

TABLE 1
Brief description of the sequences

| SEQ ID | Polypeptide/ | |
|---|---|---|
| NO. | nucleotide | Description |
| 1 | polypeptide | SAV3 |
| 2 | nucleotide | Wild type SAV3 nucleotide sequence |
| 3 | nucleotide | Codon-optimised SAV3 nucleotide sequence |
| 4 | polypeptide | SAV3 Capsid protein |
| 5 | nucleotide | Codon-optimised sequence that encodes SAV3 Capsid protein |
| 6 | polypeptide | SAV3 E3 envelope protein |
| 7 | nucleotide | Codon-optimised sequence that encodes SAV3 E3 envelope |
| | | protein |
| 8 | polypeptide | SAV3 E2 envelope protein |
| 9 | nucleotide | Codon-optimised sequence that encodes SAV3 E2 envelope |
| | | protein |
| 10 | polypeptide | SAV3 6K envelope protein |
| 11 | nucleotide | Codon-optimised sequence that encodes SAV3 6K envelope |
| | | protein |
| 12 | polypeptide | SAV3 E1 envelope protein |
| 13 | nucleotide | Codon-optimised sequence that encodes SAV3 E1 envelope |
| | | protein |
| 14 | nucleotide | 3-dbDNA SAV3 construct encoding wild-type SAV3 proteins |
| 15 | nucleotide | 5-dbDNA SAV3 codon-optimised (CO) construct |
| 16 | polypeptide | SAV3 E2 envelope protein comprising threonine residue at |
| | | position 235 |
| 17 | nucleotide | Codon-optimised sequence that encodes SAV3 E2 envelope |
| | | protein of SEQ ID NO: 16 |
| 18 | nucleotide | dbDNA SAV3-CO construct, comprising SEQ ID NO: 17 |

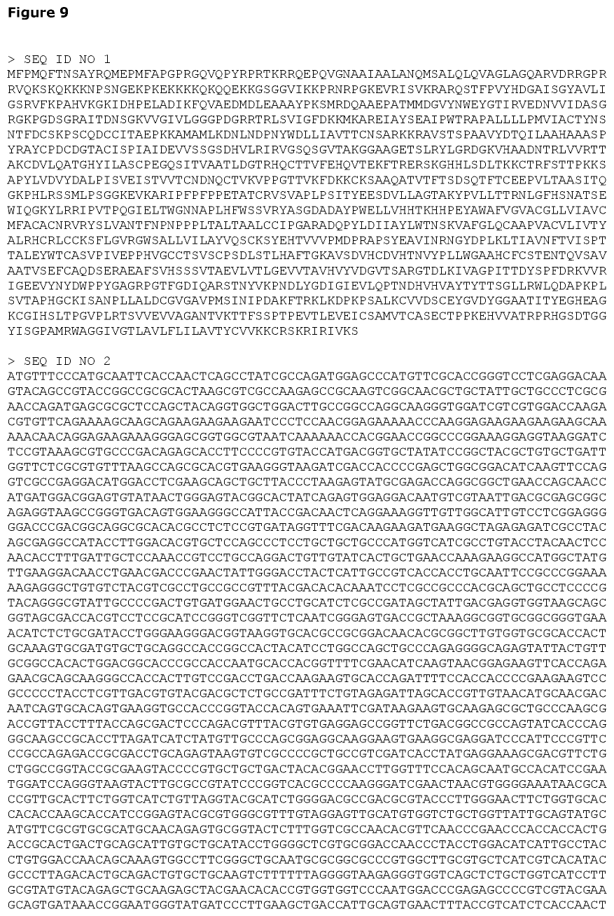

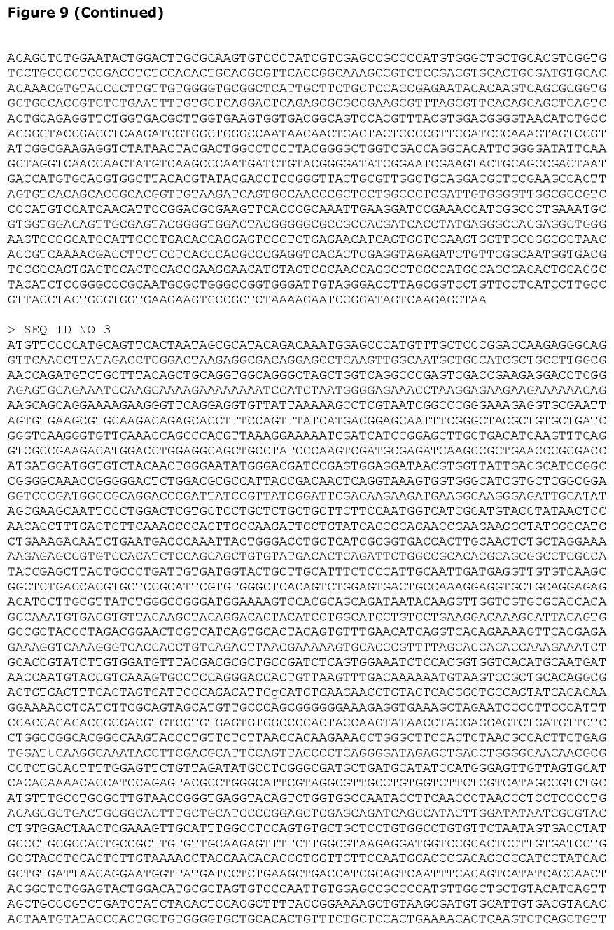

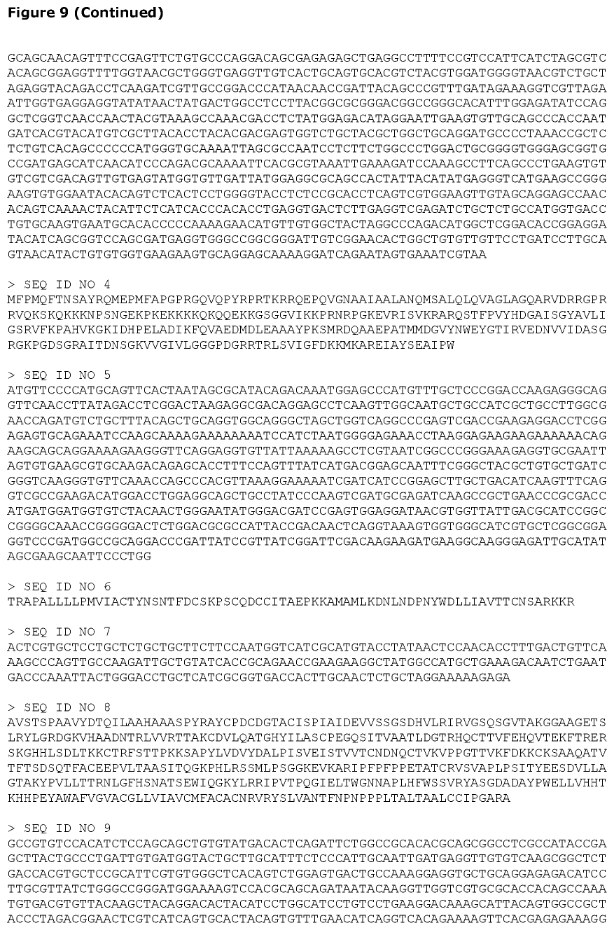

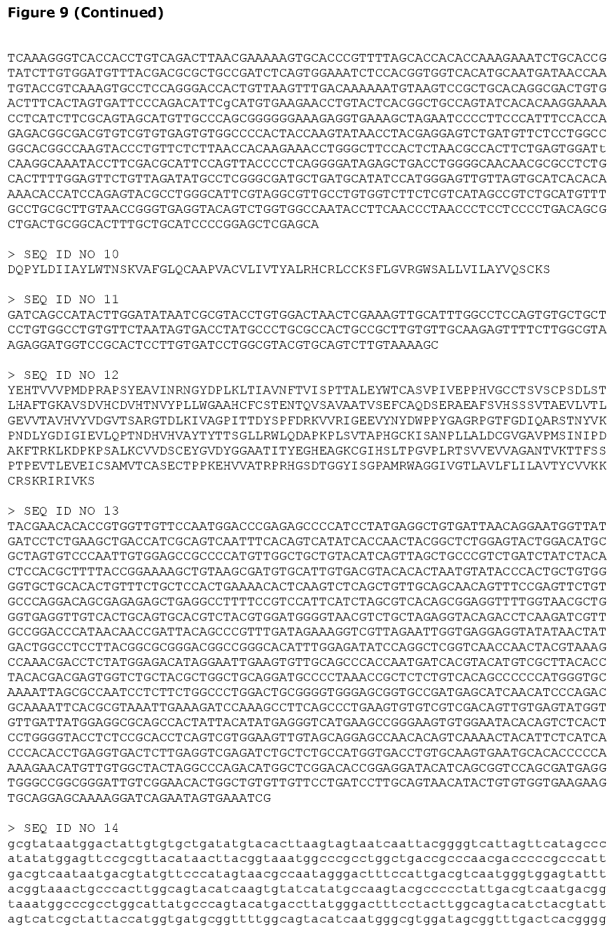

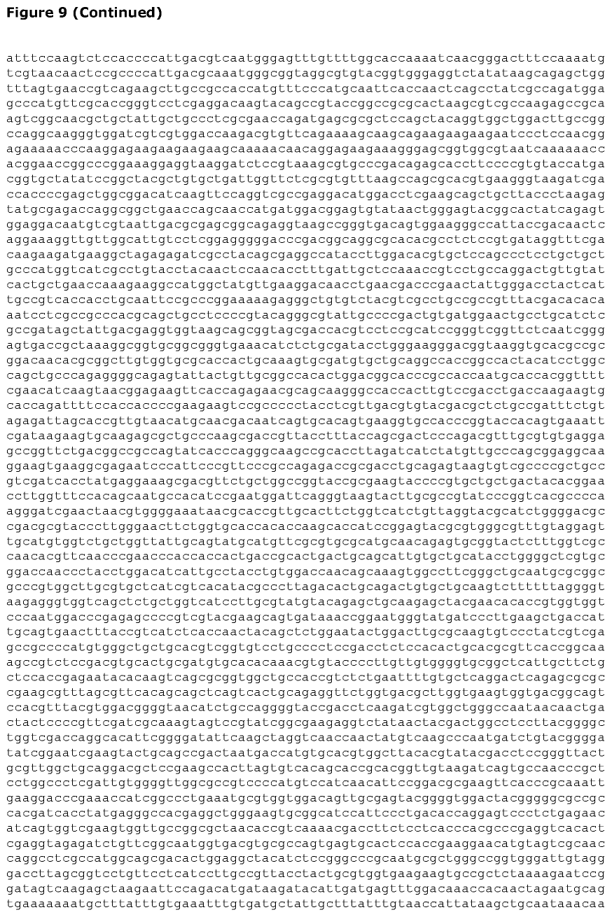

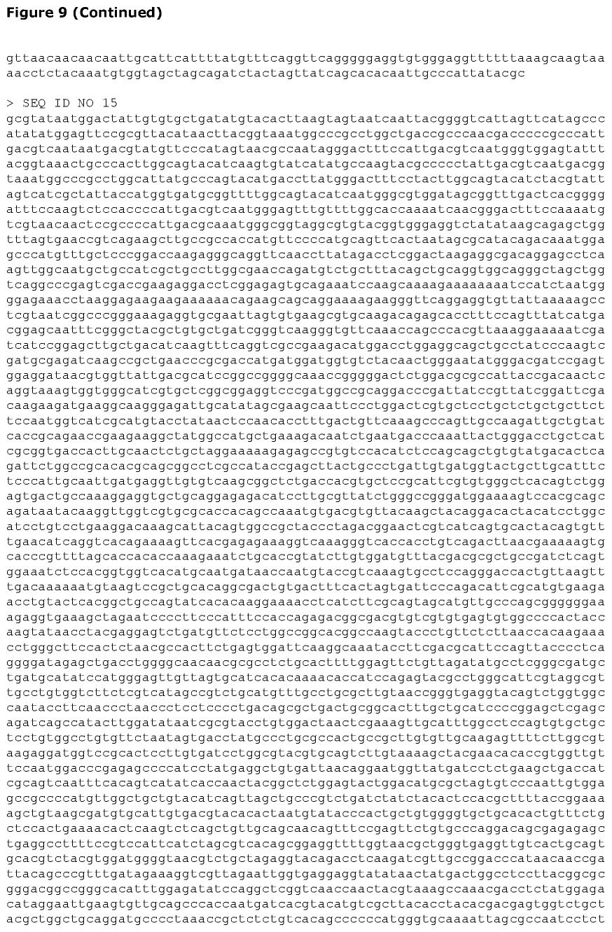

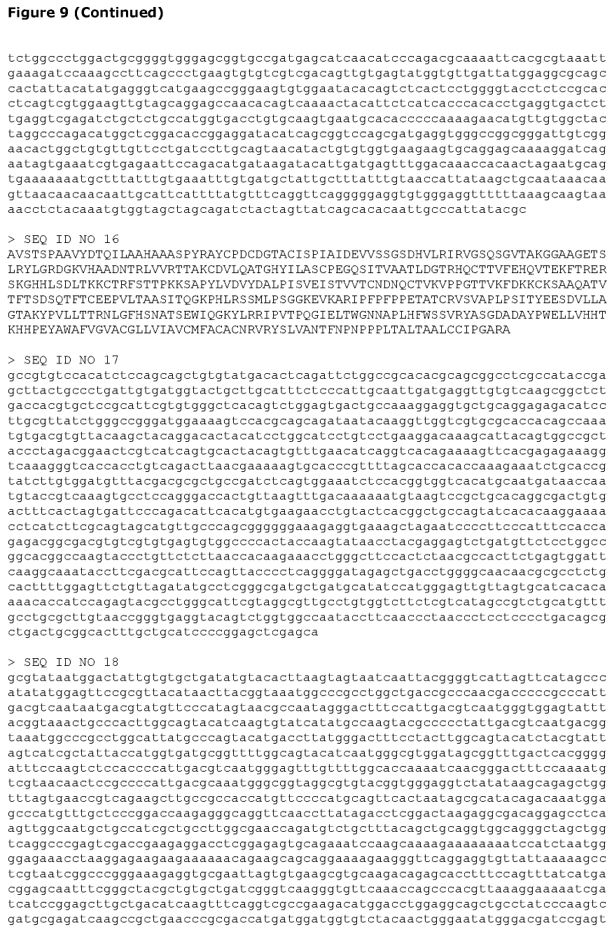

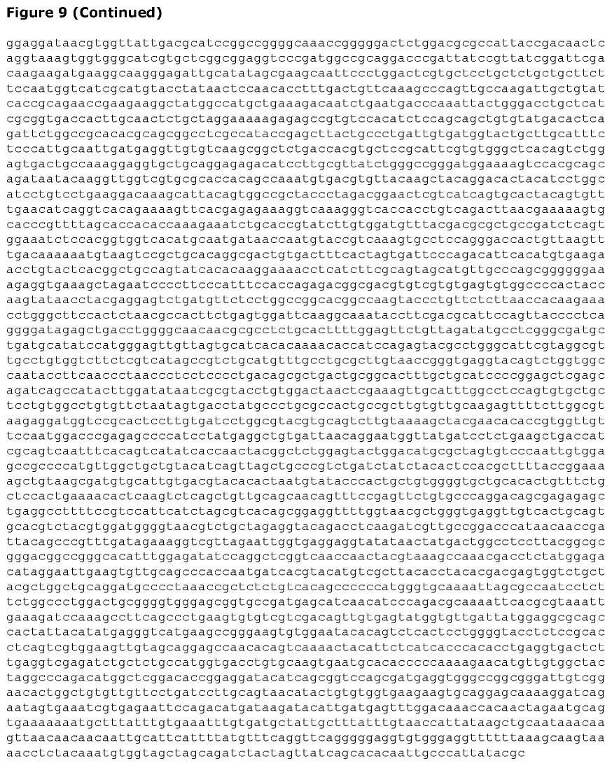

The nucleotide and protein are sequences provided in FIG. 9.

Also contemplated herein are polynucleotides comprising, consisting, or consisting essentially of nucleic acid sequences encoding fragments (e.g., truncated and partial sequences) of any one or more of the capsid and/or envelope proteins encoded by the polynucleotide described herein. Such fragments will suitably retain the ability to self-assemble into VLP under conditions that favour VLP formation. The present disclosure also extends to natural variations of the specified capsid and/or envelope proteins encoded by the polynucleotide sequences described herein. Such variants may comprise deletions, additions and/or substitutions of the polypeptide sequence that do not naturally occur in the reference protein (i.e., in nature), as long as the variant retains the ability to self-assemble to form a VLP. Suitable substitutions include those which are conservative in nature; that is, those that take place within a family of amino acids that are related in their side chains. For example, amino acids can be generally classed as: (1) acidic—aspartate and glutamate; (2) basic—lysine, arginine, histidine; (3)non-polar—alanine, valine, leucine, isoleucine, proline, phenylalanine, methionine, tryptophan; and (4) uncharged polar—glycine, asparagine, glutamine, cysteine, serine, threonine, tyrosine, and (5) aromatic—phenylalanine, tryptophan, and tyrosine.

In some embodiments, the VLP will have a quaternary structure that mimics or substantially resembles the overall structure of a corresponding wild-type, native and/or authentic salmon alphavirus particle. In this context, it will be appreciated that a salmon alphavirus VLP will be morphologically similar to a corresponding authentic, wild-type and/or native salmon alphavirus particle, since the VLP will typically have a similar conformation to native viral structural proteins. The VLP may be engineered or a product of recombinant technology and, more particularly, recombinant DNA technology, although without limitation thereto. The VLP may form spontaneously upon recombinant expression of the capsid and the at least one envelope proteins in an appropriate expression system. Methods for producing VLP will be familiar to persons skilled in the art, illustrative examples of which are discussed elsewhere herein.

The term “recombinant”, as used herein, is to be understood as meaning artificial nucleic acid structures (i.e., non-replicating cDNA or RNA; or replicons, self-replicating cDNA or RNA) which can be transcribed and/or translated in a host cell. Recombinant nucleic acid molecules may initially be inserted into a vector. Non-viral vectors such as plasmid expression vectors, or viral vectors may be used. Suitable vectors would be known to persons skilled in the art, an illustrative example of which includes an alphavirus vector. Without being bound by theory or by a particular mode of application, it is generally understood that the more structural proteins are included in the VLP, the greater the immune response is likely to be, insofar as there are more B cell and T cell epitopes to which an immune response can be raised, and additionally, the conformational veracity of the VLP is likely to be maximised. However, a sufficient immune response may nevertheless be generated where the VLP comprises only the capsid protein and the at least one envelope protein, as described herein.

The VLP may include other individual structural proteins, such as protein monomers, or dimers, or protein complexes spontaneously formed upon purification of recombinant structural proteins, such as self-assembling or intact VLP. The VLP may also be in the form of a capsid monomer, protein or peptide fragment of VLP or capsid monomer, or mixtures thereof. It is further contemplated that the VLP may further comprise a lipid envelope and/or an isolated genetic material such as DNA and/or RNA. As noted elsewhere herein, the present disclosure also extends to VLP produced using structural protein fragments or variant forms thereof, such as structural proteins that have been modified by the addition, substitution or deletion of one or more amino acids, although without limitation thereto.

In an embodiment, (a) the nucleic acid sequence encoding the salmon alphavirus capsid protein comprises the nucleic acid sequence set forth in SEQ ID NO:5; (b) the nucleic acid sequence encoding the E3 envelope protein comprises the nucleic acid sequence set forth in SEQ ID NO:7; (c) the nucleic acid sequence encoding the E2 envelope protein comprises the nucleic acid sequence set forth in SEQ ID NO:9; (d) the nucleic acid sequence encoding the 6K envelope protein comprises the nucleic acid sequence set forth in SEQ ID NO:11; and (e) the nucleic acid sequence encoding the E1 envelope protein comprises the nucleic acid sequence set forth in SEQ ID NO:13.

In an embodiment, (a) the nucleic acid sequence encoding the salmon alphavirus capsid protein consists of the nucleic acid sequence set forth in SEQ ID NO:5; (b) the nucleic acid sequence encoding the E3 envelope protein consists of the nucleic acid sequence set forth in SEQ ID NO:7; (c) the nucleic acid sequence encoding the E2 envelope protein consists of the nucleic acid sequence set forth in SEQ ID NO:9; (d) the nucleic acid sequence encoding the 6K envelope protein consists of the nucleic acid sequence set forth in SEQ ID NO:11; and (e) the nucleic acid sequence encoding the E1 envelope protein consists of the nucleic acid sequence set forth in SEQ ID NO:13.

In an embodiment, the polynucleotide comprises the nucleic acid sequence set forth in SEQ ID NO:3 or a nucleic acid sequence having 76% sequence identity thereto. By “at least 76% sequence identity” is meant 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO: 3, for example, after optimal alignment or best fit analysis. Thus, in an embodiment, the polynucleotide comprises the nucleic acid sequence having at least 76%, preferably at least 77%, preferably at least 78%, preferably at least 79%, preferably at least 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98% or more preferably at least 99% sequence identity to SEQ ID NO: 3, for example, after optimal alignment or best fit analysis. It is to be understood that a nucleic acid sequence having at least 76% sequence identity to SEQ ID NO: 3, as herein described, will suitably be a “functional variant” of the reference sequence (i.e., of SEQ ID NO: 3). It is to be understood that a “functional variant”, as used herein, means a nucleotide sequence that differs from the reference sequence to which it is being compared, which may include a natural (i.e., native) sequence or a synthetic variant thereof, yet encodes the salmon alphavirus capsid and the at least one envelope proteins capable of self-assembly to form a VLP, as described herein.

In an embodiment, the polynucleotide comprises, consists or consists essentially of the nucleic acid sequence set forth in SEQ ID NO:3. In an embodiment, the polynucleotide consists of the nucleic acid sequence set forth in SEQ ID NO:3.

In an embodiment, the nucleic acid sequence encoding the salmon alphavirus capsid protein is operatively linked to a promoter. Suitable promoters will be familiar to persons skilled in the art, illustrative examples of which include a CMV promoter, an EF1α promoter, IRFA promoter or the beta actin promoter. In another embodiment, the promoter is an IRFA promoter. In yet another embodiment, the promoter is a beta actin promoter. In an embodiment, the promoter is a CMV promoter or an EF1α promoter. Thus, in an embodiment, the promoter is a CMV promoter. In another embodiment, the promoter is an EF1α promoter. In an embodiment, the at least one nucleic acid sequence encoding the salmon alphavirus envelope protein is operatively linked to a promoter. In an embodiment, the polynucleotide further comprises a poly(A) sequence. Suitable poly(A) sequences will be known to persons skilled in the art, illustrative examples of which are described in Proudfoot N J (Genes Dev. 2011; 25(17):1770-82). In an embodiment, the polynucleotide further comprises a SV40 poly(A) sequence.

In an embodiment, the polynucleotide further comprises telomeric sequences. In an embodiment, the telomeric sequences flank both ends of the polynucleotide. The term “flank”, as used herein, typically means that the telomeric sequence is next to or adjacent the end of the polynucleotide.

In an embodiment, the polynucleotide is a closed linear polynucleotide. In an embodiment, the closed linear polynucleotide is a linear DNA covalently closed at both ends by hairpin loops.

The term “codon optimisation” as used herein refers to the replacement of certain codons with synonymous codons which allows improved expression of the resultant polypeptide or protein, while keeping the amino acid sequence of a translated protein unchanged. This is based on the discovery that although there are 64 different codons (61 codons encoding for amino acids and 3 stop codons) but only 20 different translated amino acids, that is many amino acids can be encoded for by more than one codon.

The frequency of occurrence of synonymous codons (i.e., codons that code for the same amino acid) in coding DNA can be biased in different species. Codon usage bias for a variety of organisms is known, such that a particular nucleotide sequence can be codon-optimised for expression in a host cell. Such codon degeneracy allows an identical polypeptide to be encoded by a variety of nucleotide sequences. Codon-optimised coding regions can be designed by various different methods. This optimisation may be performed using methods which are available on-line, published methods, or a company which provides codon optimising services. One codon optimising method is described, e.g., in International Patent Publication No. WO 2015/012924, which is incorporated by reference herein. Briefly, the nucleic acid sequence encoding the product is modified with synonymous codon sequences. Suitably, the entire length of the open reading frame (ORF) for the product can be modified. However, in some embodiments, only a fragment of the ORF may be altered. In some embodiments, only a few codons in the open reading frame are altered. In another embodiment, only one codon in the open reading frame is altered. By using one of these methods, one can apply the frequencies to any given polypeptide sequence, and produce a nucleic acid fragment of a codon-optimised coding region which encodes the polypeptide.

The polynucleotides described herein (also referred interchangeably herein as polynucleotide constructs) may suitably be incorporated in a vector (e.g., a recombinant vector). Suitable vectors will be familiar to persons skilled in the art, illustrative examples of which include non-viral and/or viral vectors. “Non-viral” vectors may include, for instance, plasmid vectors (e.g., compatible with bacterial, insect, yeast and/or mammalian host cells). Suitable polynucleotide constructs or vectors will be known to persons skilled in the art, illustrative examples of which include retrovirus, lentivirus, adenovirus, adeno-associated virus (AAV), herpes virus, and poxvirus, among others.

In an embodiment, a vector is employed to deliver the polynucleotide (e.g., to a cell in vitro or in vivo). Where such polynucleotides or vectors are used to induce and/or enhance an immune response, the polynucleotide or vector may also encode other proteins (e.g., co-stimulatory molecules, cytokines or chemokines) and/or be combined with other factors (e.g., exogenous cytokines). Other strategies may also be utilised to improve the efficiency of gene expression and delivery, including, for example, the use of self-replicating viral replicons, RNA replicons, in vivo electroporation, incorporation of stimulatory motifs such as CpG, sequences for targeting of the endocytic or ubiquitin-processing pathways, prime-boost regimens, proteasome-sensitive cleavage sites, and mucosal delivery systems.

Methods for preparing and using such polynucleotides, non-viral vectors, viral vectors, and variations thereof are available in the art, illustrative examples of which can be found in common molecular biology references such as Molecular Cloning: A Laboratory Manual (Sambrook et al, Cold Spring Harbor Laboratory Press, 1989), Gene Expression Technology (Methods in Enzymology, Vol. 185, edited by D. Goeddel, 1991. Academic Press, San Diego, CA), and PCR Protocols: A Guide to Methods and Applications (Innis et al, 1990. Academic Press, San Diego, CA), for instance.

In an embodiment, the polynucleotide or vector is a mini-chromosome. Examples of mini-chromosomes include PAC, BAC, YACs and artificial chromosomes). In one embodiment, the polynucleotide or vector is a closed linear polynucleotide/DNA vector. In one embodiment, the closed linear polynucleotide/vector is a ministring DNA. In one embodiment, the closed linear polynucleotide/vector is a minigene. In one embodiment, the closed linear polynucleotide/vector is a close-ended linear duplex DNA (CELiD or ceDNA). In one embodiment, the closed linear polynucleotide/vector is a doggybone (dbDNA™) DNA. dbDNA™ is a proprietary synthetic closed linear double-stranded DNA construct. Closed linear DNA is generally understood to be double-stranded DNA covalently closed at each end. The double stranded section of the DNA is therefore complementary. When denatured, closed linear DNA may form a single stranded circle. The DNA may be closed at each end by any suitable structure, including a cruciform, a hairpin or a hairpin loop, depending on preference. The end of the closed linear DNA may be composed of a non-complementary sequence, thus forcing the DNA into a single stranded configuration at the cruciform, hairpin or hairpin loop. Alternatively, the sequence can be complementary. It may be preferred that the end is formed by a portion of a target sequence for a protelomerase enzyme. A protelomerase target sequence is any DNA sequence whose presence in a DNA template allows for the enzymatic activity of protelomerase, which cuts a double stranded section of DNA and re-ligates them, leaving covalently closed ends. In general, a protelomerase target sequence is any DNA sequence whose presence in double stranded DNA template allows for its conversion into a telomeric end by the enzymatic activity of protelomerase. Known protelomerase target sequences are, in general, palindromic sequences; that is, double-stranded DNA sequence having two-fold rotational symmetry, or a perfect inverted repeat. The closed linear DNA may have a portion of a protelomerase target sequence at one or both ends. This portion remains after the protelomerase has cleaved and re-ligated its cognate sequence. The portion of the protelomerase target sequence at each end can be the same or different; for instance, the portion may be generated from protelomerase target sequences that have different cognate enzymes, or the same cognate enzyme. Closed linear DNA constructed via the action of various protelomerase enzymes have been previously disclosed in WO2010/086626, WO2012/017210 and WO2016/132129, the entire contents of which are incorporated herein by reference. Closed linear DNA constructed using in vitro DNA amplification followed by cleavage with a protelomerase enzyme has the advantage that the closed linear DNA is produced in an in vitro, cell-free environment, and can be scaled up for commercial production.

In an embodiment, the closed linear polynucleotide comprises covalently closed ends also described as hairpin loops, where base-pairing between complementary DNA strands is not present. The hairpin loops join the ends of complementary DNA strands. In some embodiments, the closed linear polynucleotide construct comprises telomeric sequences. In an embodiment, the telomeric sequences are situated such that they cap the the closed linear polynucleotide. In another embodiment, the telomeric sequences flank both ends of the promoter-linked polynucleotide.

In an embodiment, the polynucleotide construct comprises an expression cassette. A nucleic acid cassette or expression cassette is intended to mean a nucleic acid sequence designed to introduce a nucleic acid sequence, typically a heterologous nucleic acid sequence (e.g., the nucleic acid construct as described herein) into a vector. The expression cassette may include a terminal restriction enzyme linker (i.e., Restriction Enzyme recognition nucleotides) at each end of the sequence of the cassette to facilitate insertion of the nucleic acid sequence or sequences of interest. The terminal restriction enzyme linkers at each end may be the same or different terminal restriction enzyme linkers. In some embodiments, the terminal restriction enzyme linkers may include rare restriction enzyme recognition/cleavage sequences, such that unintended digestion of the nucleic acid or the alphavirus genome into which the cassette is to be introduced does not occur. Suitable terminal restriction enzyme linkers would be known to persons skilled in the art.

In an embodiment, the nucleic acid sequences encoding of the peptide sequences disclosed herein are codon optimised for expression in the host cell. Suitable methods of codon optimisation will be familiar to persons skilled in the art, illustrative examples of which are described in Mauro and Chappell (2014; Trends Mol. Med., 20(11):604-613) and in Mauro (2018; BioDrugs, 32:69-81), the contents of which are incorporated herein by reference.

The present disclosure also extends to a cell comprising the polynucleotide described herein. Suitable host cells permissive for viruses and for the formation of VLP will be familiar to persons skilled in the art, illustrative examples of which are described in Sarkar et al. (Korean J. Microbiol. 2019; 55(4):327-343), Santi et al. (Methods. 2006; 40(1): 66-76), Roldio et al. (Expert Rev. Vaccines; 2010; 9(10): 1149-1176) and Ghislain Masavuli et al. (Front Microbiol. 2017; 8: 2413), and include bacteria, yeast, fungi, plant, insect, mammalian and avian cells. Reference is also made to “Short Protocols in Molecular Biology, 5th Edition, 2 Volume Set: A Compendium of Methods from Current Protocols in Molecular Biology” (by Frederick M. Ausubel (author, editor), Roger Brent (editor), Robert E. Kingston (editor), David D. Moore (editor), J. G. Seidman (editor), John A. Smith (editor), Kevin Struhl (editor), J Wiley & Sons, London).

In an embodiment, the cell is a bacterium. Suitable virus-permissive bacteria will be familiar to persons skilled in the art, an illustrative example of which includes Escherichia coli (Huang et al., NPJ Vaccines. 2017; 2:3).

In an embodiment, the cell is a yeast cell. Suitable virus permissive yeast cells will be familiar to persons skilled in the art, illustrative examples of which are described in Kumar and Kumar (FEMS Yeast Research, 2019; 19(2): foz007) and include Pichia Pastoris (Kim and Kim, Letters Appl. Micro., 2017; 64(2):111-123), Saccharomyces cerevisiae (Zhao et al. Appl Microbiol Biotechnol. 2013; 97(24):10445-52) and Hansenula polymorpha (Wetzel et al. J Biotechnol. 2019; 20; 306:203-212).

In an embodiment, the cell is a plant cell. Suitable virus permissive plant cells will be familiar to persons skilled in the art, illustrative examples of which are described in Makarkov et al. (npj Vaccines; 2019; 4(17)) and Santi et al. (Methods. 2006; 40(1): 66-76), and include Nicotiana tabacum and Arabidopsis thaliana (Greco et al. Vaccine. 2007; 28;25(49):8228-40), Samsun N N and Solanum tuberosum cv. Solara.

In an embodiment, the cell is an insect cell. Suitable virus permissive insect cells will be familiar to persons skilled in the art, illustrative examples of which include Spodoptera frugiperda (sf9; Wagner et al. PLoS One. 2014; 9(4):e94401), Trichoplusia ni (BTI-TN5B1-4; Krammer et al., Mol Biotechnol. 2010; 45(3):226-234) and Drosophila Schneider 2 (S2; Park et al., J Virol Methods. 2018; 261:156-159).

In an embodiment, the cell is a fish cell. In an embodiment, the cell is a salmon cell. Fish cells suitably allow expression of the peptides encoded by the polynucleotide described herein, including because the polynucleotides described herein are codon-optimised for expression in fish.

The present disclosure also extends to a composition comprising the polynucleotide, the cell or the salmon alphavirus-like particle described herein.

The present disclosure also extends to a salmon alphavirus-like particle (VLP) encoded by the polynucleotide described herein.

The present disclosure also extends to methods of manufacturing the VLP described herein. Thus, in an aspect disclosed herein, there is provided a method of producing a salmon alphavirus-like particle (VLP), the method comprising:
(a) introducing the polynucleotide described herein into a host cell; and
(b) culturing the host cell of (a) under conditions and for a period of time sufficient for the host cell to produce the VLP.

Suitable culture methods and conditions/times suitable for producing VLP will be familiar to persons skilled in the art, understanding that the method steps and conditions/times will likely vary depending, for example, on the type of host cell employed. Illustrative examples of suitable methods of production are set out elsewhere herein and also described in Cheng and Mukhopadhyay (Virology, 2011; 413(2):153-160), Charlton Hume et al. (ibid), Ghislain Masavuli et al. (ibid) and Vacher et al. (Mol. Pharmaceutics 2013; 10:1596-1609).

The recombinant structural virus proteins (e.g., capsid and envelope proteins) used to prepare the VLP described herein can be readily prepared by standard genetic engineering techniques by the skilled person provided with the sequence of the wild-type protein. Methods of genetically engineering proteins are well known in the art, illustrative examples of which are described, for example, in Ausubel et al. (1994 and updates) Current Protocols in Molecular Biology, John Wiley & Sons, New York). Isolation and cloning of the nucleic acid sequence encoding the structural proteins can be achieved using standard techniques (see, e.g., Ausubel et al., ibid.). For example, the nucleic acid sequence can be obtained directly from the virus by extracting RNA by standard techniques and then synthesising cDNA from the RNA template (e.g., by RT-PCR). The nucleic acid sequence encoding the structural proteins is then inserted directly or after one or more subcloning steps into a suitable expression vector. Persons skilled in the art will understand that the precise vector used is not critical. Illustrative examples of suitable vectors include plasmids, phagemids, cosmids, bacteriophage, baculoviruses, retroviruses or DNA viruses. The structural protein(s) can then be expressed and purified as described in more detail below.

Alternatively, the nucleic acid sequence encoding the structural protein(s) can be further engineered to introduce one or more mutations, such as those described above, by standard in vitro site-directed mutagenesis techniques known to persons skilled in the art. The nucleic acid sequence encoding the structural proteins may also be rationally designed and synthesised. Mutations can be introduced by deletion, insertion, substitution, inversion, or a combination thereof, of one or more of the appropriate nucleotides making up the coding sequence. This can also be achieved, for example, by PCR based techniques for which primers are designed that incorporate one or more nucleotide mismatches, insertions or deletions. The presence of the mutation can be verified by a number of standard techniques, for example by restriction analysis or by DNA sequencing.

Virus proteins may also be engineered to produce fusion proteins comprising one or more immunogens fused to virus coat protein. Methods for making fusion proteins are well known to those skilled in the art. DNA sequences encoding a fusion protein can be inserted into a suitable expression vector as noted above. Persons skilled in the art will appreciate that the DNA encoding the coat protein or fusion protein can be altered in various ways without affecting the activity of the encoded protein. For example, variations in DNA sequence may be used to optimise for codon preference in a host cell used to express the protein, or may contain other sequence changes that facilitate expression.

As noted elsewhere herein, the expression vector may further include regulatory elements, such as transcriptional elements, required for efficient transcription of the DNA sequence encoding the coat or fusion protein. Illustrative examples of suitable regulatory elements that can be incorporated into the vector include promoters, enhancers, terminators, and polyadenylation signals. The present disclosure therefore also provides vectors comprising a regulatory element operatively linked to one or more nucleic acid sequences encoding the VLP described herein. Persons skilled in the art will appreciate that selection of suitable regulatory elements may be dependent on the host cell chosen for expression of the VLP and that such regulatory elements may be derived from a variety of sources, including bacterial, fungal, viral, plant, mammalian or insect genes. The expression vector may additionally contain heterologous nucleic acid sequences that facilitate the purification of the expressed VLP. Illustrative examples of suitable heterologous nucleic acid sequences include affinity tags such as metal-affinity tags, histidine tags, avidin/streptavidin encoding sequences, glutathione-S-transferase (GST) encoding sequences and biotin encoding sequences. The amino acids corresponding to expression of the nucleic acids can be removed from the expressed VLP prior to use, including clinical use, as described elsewhere herein. Alternatively, the amino acids corresponding to expression of heterologous nucleic acid sequences can be retained on the VLP if they do not interfere with subsequent use. The expression vector can be introduced into a suitable host cell by one of a variety of methods known in the art, illustrative examples of which include stable or transient transfection, lipofection, electroporation, and infection with recombinant viral vectors. One skilled in the art will understand that selection of the appropriate host cell for expression of the viral structural proteins (the capsid and the envelope protein(s)) may be dependent upon the vector chosen. Illustrative examples of suitable host cells are described elsewhere herein and include bacterial, yeast, insect, plant and mammalian cells.

When the polynucleotide sequences described herein are introduced into a host cell and subsequently expressed at the necessary level, the VLP will suitably assemble and can then be released from the cell into the culture media, where it can be further processed (e.g., purified), if necessary, having regard to the intended use.

Depending on the expression system and host cell selected, the VLP, as herein defined, are typically produced by growing the host cell transformed by the expression vector under conditions whereby the viral proteins encoded by the polypeptide/vector are expressed and VLP can be formed. The selection of the appropriate growth conditions will be familiar to persons skilled in the art.

The VLP can be isolated (or substantially purified) using methods that preserve the integrity thereof, such as, by density gradient centrifugation, e.g., sucrose gradients, PEG-precipitation, pelleting, and the like (see, e.g., Kirnbauer et al. J. Virol. (1993) 67:6929-6936), as well as standard purification techniques including, e.g., ion exchange and gel filtration chromatography. For example, a composition or preparation comprising an isolated VLP prepared according to the method of the present disclosure may comprise at least 65%, at least 70%, at least 75%, at least 80%, at least 85%, at least 90%, at least 95%, at least 96%, at least 98%, at least 99% or 100% of an isolated VLP, as measured by methods known to persons skilled in the art.

The presence of the VLP can be detected using conventional techniques known in the art, such as by electron microscopy, atomic force microscopy, biophysical characterisation, and the like (see, e.g., Baker et al., Biophys. J. (1991) 60:1445-1456; and Hagensee et al., J. Virol. (1994) 68:4503-4505). For example, the VLP can be isolated by density gradient centrifugation and/or identified by characteristic density banding. Alternatively, cryoelectron microscopy can be performed on vitrified aqueous samples of the VLP preparation in question, and images recorded under appropriate exposure conditions.

The present disclosure also extends to a VLP produced by the methods described herein and a vaccine composition comprising the vaccine constructs and/or VLP, as described herein.

### Methods of Treating or Protecting Against Salmon Alphavirus Infection

The present disclosure also extends to a method for treating or protecting against salmon alphavirus infection in a subject, the method comprising administering to a subject in need thereof the polynucleotide, the salmon alphavirus-like particle or the composition described herein.

In an embodiment, the polynucleotide, the salmon alphavirus-like particle or the composition described herein is administered to the subject by intramuscular injection.

In an embodiment, the method described herein comprises administering to the subject the polynucleotide construct in an amount of from about 0.1 microgram to about 20 micrograms. In an embodiment, the method described herein comprises administering to the subject the polynucleotide construct in an amount of from about 0.2 microgram to about 18 micrograms. In an embodiment, the method described herein comprises administering to the subject the polynucleotide construct in an amount of from about 0.3 microgram to about 15 micrograms. In a preferred embodiment, the method described herein comprises administering to the subject the polynucleotide construct in an amount of from about 0.5 microgram to about 10 micrograms. In another preferred embodiment, the method described herein comprises administering to the subject the polynucleotide construct in an amount of from about 0.5 microgram to about 5 micrograms. In another preferred embodiment, the method described herein comprises administering to the subject the polynucleotide construct in an amount of from about 1 microgram to about 5 micrograms.

In an embodiment, the subject has, or is at risk of developing, salmonoid pancreatic disease.

The present disclosure also extends to a use of the polynucleotide, the cell or the salmon alphavirus-like particle described herein, in the manufacture of a vaccine composition for treating or protecting against salmon alphavirus infection in a subject.

The present disclosure also extends to the polynucleotide described herein for use in treating or preventing against salmon alphavirus infection in a subject.

The present disclosure also extends to a method of inducing an immune response against a salmonid alphavirus in a subject, the method comprising administering to a subject in need thereof the polynucleotide, the salmon alphavirus-like particle or the composition described herein.

In an embodiment, the salmonid alphavirus is a salmon alphavirus that results in salmon pancreas disease or sleeping disease in salmonid fish. In an embodiment, the salmonid alphavirus is a subtype selected from the group consisting of SAV1, 2, 3, 4, 5 and 6. In an embodiment, the salmonid alphavirus is a SAV1 subtype. In an embodiment, the salmonid alphavirus is a SAV4 subtype. In an embodiment, the salmonid alphavirus is a SAV5 subtype. In an embodiment, the salmonid alphavirus is a SAV6 subtype. In an embodiment, the salmonid alphavirus is a SAV2 subtype. In another embodiment, the salmonid alphavirus is a SAV3 subtype.

The present disclosure also extends to polynucleotides comprising a nucleic acid sequence encoding combinations of salmon alphavirus capsid proteins and one or more salmon alphavirus envelope proteins, including where the capsid and the one or more salmon alphavirus envelope proteins are from different salmon alphavirus subtypes, as described herein. Suitable combinations may comprise capsid and envelope proteins of the same salmon alphavirus subtype (e.g., subtypes 1-6), or they may comprise capsid and envelope proteins of two or more different salmon alphavirus subtypes. In an embodiment, the combination comprises a capsid protein of salmon alphavirus subtype 1 and one or more envelope proteins of a salmon alphavirus subtype selected from the group consisting of subtypes 2-6, illustrative examples of which are described herein. In another embodiment, the combination comprises a capsid protein of salmon alphavirus subtype 2 and one or more envelope proteins of a salmon alphavirus subtype selected from the group consisting of subtypes 1 and 3-6, illustrative examples of which are described herein. In another embodiment, the combination comprises a capsid protein of salmon alphavirus subtype 3 and one or more envelope proteins of a salmon alphavirus subtype selected from the group consisting of subtypes 1, 2 and 4-6, illustrative examples of which are described herein. In another embodiment, the combination comprises a capsid protein of salmon alphavirus subtype 4 and one or more envelope proteins of a salmon alphavirus subtype selected from the group consisting of subtypes 1-3, 5 and 6, illustrative examples of which are described herein. In another embodiment, the combination comprises a capsid protein of salmon alphavirus subtype 5 and one or more envelope proteins of a salmon alphavirus subtype selected from the group consisting of subtypes 1-4 and 6, illustrative examples of which are described herein. In another embodiment, the combination comprises a capsid protein of salmon alphavirus subtype 6 and one or more envelope proteins of a salmon alphavirus subtype selected from the group consisting of subtypes 1-5, illustrative examples of which are described herein.

In another embodiment disclosed herein, there is provided a polynucleotide comprising a nucleic acid sequence encoding a combination of two or more salmon alphavirus envelope proteins from different salmon alphavirus subtypes, as described herein. In an embodiment, the combination comprises two or more envelope proteins of a salmon alphavirus subtype selected from the group consisting of subtypes 2-6, illustrative examples of which are described herein. In another embodiment, the combination comprises two or more envelope proteins of a salmon alphavirus subtype selected from the group consisting of subtypes 1 and 3-6, illustrative examples of which are described herein. In another embodiment, the combination comprises two or more envelope proteins of a salmon alphavirus subtype selected from the group consisting of subtypes 1, 2 and 4-6, illustrative examples of which are described herein. In another embodiment, the combination comprises two or more envelope proteins of a salmon alphavirus subtype selected from the group consisting of subtypes 1-3, 5 and 6, illustrative examples of which are described herein. In another embodiment, the combination comprises two or more envelope proteins of a salmon alphavirus subtype selected from the group consisting of subtypes 1-4 and 6, illustrative examples of which are described herein. In another embodiment, the combination comprises two or more envelope proteins of a salmon alphavirus subtype selected from the group consisting of subtypes 1-5, illustrative examples of which are described herein.

In an embodiment, the immune response comprises an antibody response. In another embodiment, the antibody response is the induction of antibodies capable of binding specifically to and neutralising the salmon alphavirus.

In an embodiment, the antibodies are capable of binding specifically to and neutralising SAV2 and SAV3 subtypes.

For administration to a host or subject (e.g., salmon), the vaccine construct, VLP and/or compositions described herein can be formulated for administration by a variety of routes. For example, the vaccine construct, VLP and/or compositions described herein can be formulated for oral, topical, rectal or parenteral administration or for administration by inhalation, intranasally or spray. The term “parenteral”, as used herein, includes subcutaneous injections, intradermal, intravenous, intramuscular, intrathecal, intrasternal injection and infusion techniques. In an embodiment, the vaccine construct, the VLP or the composition described herein is administered to the subject by intramuscular injection. In an embodiment, the vaccine construct, the VLP or the composition described herein is administered to the subject by intraperitoneal injection. In another embodiment, the vaccine construct, the VLP or the composition described herein is administered to the subject through immersion (i.e., via uptake through the gills), by introducing the vaccine construct, the VLP or the composition described herein into the water in which the fish inhabits.

The vaccine construct, VLP and/or compositions described herein will suitably comprise a therapeutically effective amount of the vaccine construct and/or VLP. The phrase “therapeutically effective amount” typically means an amount of the vaccine construct and/or VLP, as described herein, necessary to attain the desired response, for example, the inducement of an immune response to the target (i.e., to the salmon alphavirus). Typically, the appropriate dosage of the vaccine construct and/or VLP, as described herein, may depend on a variety of factors including, but not limited to, a subject's physical characteristics (e.g., age, weight, sex), whether the vaccine construct and/or VLP, as described herein, is being used as single agent or as part of adjuvant therapy, the progression (i.e., pathological state) of any underlying virus infection or disease, and other factors that may be recognised by persons skilled in the art. Various general considerations that may be considered when determining, for example, an appropriate dosage of the vaccine composition (see, e.g., in Gennaro (2000) “Remington: The Science and Practice of Pharmacy”, 20th edition, Lippincott, Williams, & Wilkins; and Gilman et al., (Eds), (1990), “Goodman And Gilman's: The Pharmacological Bases of Therapeutics”, Pergamon Press). It is expected that the amount will fall in a relatively broad range that can be determined through methods known to persons skilled in the art. Illustrative examples of a suitable therapeutically effective amount of vaccine construct and/or VLP for administration to a subject include from about 0.1 microgram to about 20 micrograms, from about 0.2 microgram to about 18 micrograms, from about 0.3 microgram to about 15 micrograms, from about 0.5 microgram to about 10 micrograms, from about 0.5 microgram to about 5 micrograms, or from about 1 microgram to about 5 micrograms. Dosage regimes may be adjusted to provide the optimum therapeutic response. For example, several divided doses may be administered daily, weekly, monthly or other suitable time intervals, or the dose may be proportionally reduced as indicated by the exigencies of the situation.

In an embodiment, a therapeutically effective amount of the vaccine construct and/or VLP, as described herein, as defined herein, is effective to induce an immune response to the target, irrespective of genotype.

As described elsewhere herein, the terms “immune response”, “immunological response” and the like are typically used herein to refer to the development in a subject of a humoral and/or a cellular immune response to the target. A “humoral immune response” typically refers to an immune response mediated by antibody molecules, while a “cellular immune response” is typically mediated by T-lymphocytes and/or other white blood cells. In a non-limiting example, the vaccine construct and/or VLP, as described herein, when administered to a subject induces an immune response selected from one or more of a neutralising antibody response, a cytotoxic T lymphocyte (CTL) response, a natural killer T cell response and/or a helper T lymphocyte (e.g., CD4⁺ T cell) response and innate immune response to the target antigen.

Methods for measuring an immune response will be known to persons skilled in the art, illustrative examples of which include plaque-reduction neutralisation assay, micro-neutralisation assay, solid-phase heterogeneous assays (e.g., enzyme-linked immunosorbent assay), solution phase assays (e.g., electrochemiluminescence assay), Western immunoblot, amplified luminescent proximity homogeneous assays, flow cytometry, intracellular cytokine staining, functional T-cell assays including suppressor T-cell assays, functional B-cell assays, functional monocyte-macrophage assays, dendritic and reticular endothelial cell assays, measurement of NK or NKT cell responses, oxidative burst assays, cytotoxic-specific cell lysis assays, pentamer binding assays, and phagocytosis and apoptosis evaluation.

As described elsewhere herein, the vaccine construct and/or VLP, as described herein, can be administered to a subject in need thereof by any suitable route of administration, including administration of the composition orally, nasally, nasopharyngeally, parenterally, enterically, gastrically, topically, transdermally, subcutaneously, intramuscularly, intradermally, in tablet, solid, powdered, liquid, aerosol form, locally or systemically, with or without added excipients. Suitable methods for preparing parenterally administrable compositions will be known or apparent to those skilled in the art and are described in more detail in such publications as Remington's Pharmaceutical Science, 15th ed., Mack Publishing Company, Easton, Pa. (1980). In some instances, administration is accomplished by intramuscular injection of the vaccine construct and/or VLP. In general, the vaccine construct and/or VLP, as described herein, can be administered in a manner compatible with the route of administration and physical characteristics of the recipient (including health status) and in such a way that it elicits the desired effect(s) (e.g., the induction of a protective immune response against the target).

The vaccine construct and/or VLP, as described herein, may be administered to a recipient in isolation or in combination with other additional therapeutic agent(s). In embodiments where a pharmaceutical composition comprising the vaccine construct and/or VLP, as described herein, is formulated for administration with additional therapeutic agent(s), the administration may be simultaneous or sequential (i.e., administration of the vaccine construct and/or VLP is followed by administration of the additional agent(s) or vice versa). Thus, here two or more entities are administered to a subject “in conjunction”, they may be administered in a single composition at the same time, or in separate compositions at the same time, or in separate compositions separated in time.

In a non-limiting example, the vaccine construct and/or VLP, as described herein, may be administered in conjunction with another antiviral agent. An “antiviral agent” typically means an agent which, when administered to a subject, is capable of significantly reducing the virus titre in the blood or serum either directly (e.g., by inhibiting a viral enzyme activity) or indirectly (e.g., via modulation of the antiviral responses of a host cell), either transiently or in a sustained way.

In an embodiment, the vaccine construct and/or VLP, as described herein, may suitably be given in an appropriate single dosage in order to elicit an immune response. In other embodiments, the initial dose may be followed by boosting dose. The boosting dose may comprise the same vaccine construct and/or VLP as the initial (priming) dose, whether at an equivalent dose (e.g., the same or similar dose), a lower dose or a higher dose as compared to the initial dose.

The administration regime need not differ from any other generally accepted vaccination programs. For instance, a single administration in an amount sufficient to elicit an effective immune response may be used. Alternatively, as noted above, other regimes of initial administration of the complex followed by boosting, including as described above. Boosting may occur at times that take place well after the initial administration if the immune response (as measured, e.g., by antibody titres) falls below acceptable levels.

Alternatively, or in addition, the vaccine construct and/or VLP, as described herein, can be used in combination an additional immunopotentiator or adjuvant to enhance an immune response in the subject. The present disclosure therefore extends to compositions further comprising an immunopotentiator or adjuvant. Preferably, the immunopotentiator or adjuvant is administered concomitantly with the polynucleotide and/or VLP, as described herein. The immunopotentiator or adjuvant can be administered prior or subsequently to the polynucleotide and/or VLP, as described herein, depending on the need as can be suitably determined by persons skilled in the art. The term “immunopotentiator,” as used herein, is intended to mean a substance that, when mixed with an immunogen, elicits a greater immune response than the immunogen alone. For example, an immunopotentiator can enhance immunogenicity and provide a superior immune response. If desired, an adjuvant or other active ingredient optionally may be included in the compositions. In an embodiment, the polynucleotide and/or VLP, or compositions thereof as described herein, are capable of efficiently potentiating an immune response in the absence of an additional adjuvant.

Suitable immunopotentiators or adjuvants will be familiar to persons skilled in the art, illustrative examples of which include mineral and non-mineral oil based emulsions, such as water in oil, oil in water, or water/oil/water (WOW) formulations. Another example of an emulsion based adjuvant includes squalene-based, oil-in-water nano-emulsion vaccine adjuvants (e.g. AddaVax (Invivogen)). Other illustrative examples of which include 1018 ISS, aluminum salts, AMPLIVAX®, AS15, BCG, CP-870,893, CpG7909, CyaA, dSLIM, flagellin or TLRS ligands derived from flagellin, FLT3 ligand, GM-CSF, IC30, IC31, Imiquimod (ALDARA^{T}M), resiquimod, ImuFact IMP321, Interleukins such as IL-2, IL-12, IL-18, IL-21, Interferon-alpha or -beta or -gamma or pegylated derivatives thereof, IS Patch, ISS, ISCOMATRIX, ISCOMs, JuvImmune™, LipoVac, MALP2, MF59, monophosphoryl lipid A, Montanide IMS 1312, Montanide ISA 206, Montanide ISA 50V, Montanide ISA-51, other suitable water-in-oil and oil-in-water emulsions (e.g., AddaVax), OK-432, OM-174, OM-197-MP-EC, ONTAK, OspA, PepTel™. vector system, poly(lactid co-glycolid) [PLG]-based and dextran microparticles, talactoferrin SRL172, YF-17D, VEGF trap, R848, beta-glucan, Pam3Cys, Aquila's QS21 stimulon, which is derived from saponin, mycobacterial extracts and synthetic bacterial cell wall mimics, and other proprietary adjuvants such as Ribi's Detox, Quil, or Superfos. Other examples of adjuvants include Freund's or GM-CSF.

Immunopotentiating cytokines may also be used. Several cytokines have been directly linked to influencing dendritic cell migration to lymphoid tissues (e.g., TNF-), accelerating the maturation of dendritic cells into efficient antigen-presenting cells for T-lymphocytes (e.g., GM-CSF, IL-1 and IL-4) (see, e.g., U.S. Pat. No. 5,849,589, specifically incorporated herein by reference in its entirety) and acting as immunoadjuvants (e.g., IL-12, IL-15, IL-23, IL-7, IL-18, IFN-alpha. IFN-beta). These immunopotentiating cytokines can be included as polypeptide molecules in the compositions, or be encoded for by nucleic acids, which can be co-administered with the polynucleotide and/or VLP, as described herein, or be included in the polynucleotide as described herein.

CpG immunostimulatory oligonucleotides have also been reported to have immunopotentiating effects. Without being bound by theory or by a mode of application, CpG oligonucleotides act by activating the innate (non-adaptive) immune system via Toll-like receptors (TLR), mainly TLR9. CpG-triggered TLR9 activation enhances antigen-specific humoral and cellular responses to a wide variety of antigens, including peptide or protein antigens, live or killed viruses, dendritic cell vaccines, autologous cellular vaccines and polysaccharide conjugates in both prophylactic and therapeutic vaccines. More importantly, it can enhance dendritic cell maturation and differentiation, resulting in enhanced activation of Th1 cells and strong cytotoxic T-lymphocyte (CTL) generation, even in the absence of CD4 T cell help. The Th1 bias induced by TLR9 stimulation is maintained even in the presence of vaccine adjuvants such as alum or incomplete Freund's adjuvant (IFA) that normally promote a Th2 bias. CpG oligonucleotides show even greater adjuvant activity when formulated or co-administered with other adjuvants or in formulations such as microparticles, nanoparticles, lipid emulsions or similar formulations, which are especially necessary for inducing a strong response when the antigen is relatively weak. They also accelerate the immune response and enable the antigen doses to be reduced by approximately two orders of magnitude, with comparable antibody responses to the full-dose vaccine without CpG. U.S. Pat. No. 6,406,705 describes the combined use of CpG oligonucleotides, non-nucleic acid adjuvants and an antigen to induce an antigen-specific immune response. A CpG TLR9 antagonist is dSLIM (double Stem Loop Immunomodulator) by Mologen (Berlin, Germany) which is a preferred component of the pharmaceutical composition of the present invention. Other TLR binding molecules such as RNA binding TLR 7, TLR 8 and/or TLR 9 may also be used.

Other illustrative examples of suitable adjuvants include chemically modified CpGs (e.g., CpR, Idera), dsRNA analogues such as Poly(I:C) and derivates thereof (e.g. AmpliGen®, Hiltonol®, poly-(ICLC), poly(IC-R), poly(I:C12U), non-CpG bacterial DNA or RNA as well as immunoactive small molecules and antibodies such as cyclophosphamide, sunitinib, Bevacizumab®, celebrex, NCX-4016, sildenafil, tadalafil, vardenafil, sorafenib, temozolomide, temsirolimus, XL-999, CP-547632, pazopanib, VEGF Trap, ZD2171, AZD2171, anti-CTLA4, other antibodies targeting key structures of the immune system, which may act therapeutically and/or as an adjuvant. The amounts and concentrations of adjuvants and additives useful in the context of the present invention can readily be determined by the skilled artisan without undue experimentation.

The vaccine construct and/or VLP, or compositions thereof as described herein can further comprise one or more immunostimulatory or adjuvanting lipids. Suitable immunostimulatory lipids will be familiar to persons skilled in the art, illustrative examples of which include glycolipids and phospholipids. In an embodiment, the immunostimulatory lipid is an immunostimulatory glycolipid or an immunostimulatory phospholipid. The immunostimulatory lipid may be a naturally-occurring lipid or it may be synthetically derived (synthesised). In some embodiments, the immunostimulatory lipid is a synthetic lipid. In some embodiments, the synthetic lipid may resemble, in part or in whole, a naturally-occurring lipid. Thus, also contemplated herein are immunostimulatory lipid analogues; that is, synthetic immunostimulatory lipids that resemble, at least in part, naturally-occurring lipids and yet are still capable of acting as an adjuvant to potentiate a host's immune response to an immunogen. Suitable immunostimulatory lipid analogues will be familiar to persons skilled in the art, illustrative examples of which are described in US patent publication nos. 20200009165 and 20190358318, Maeda et al. (Vaccine; 1989; 7(3):275-281); Jiang et al. (Carbohydr. Res., 2007; 342(6):784-796); Foster et al. (J. Med. Chem., 2018; 61(3):1045-1060), the contents of which are incorporated herein by reference in their entirety.

The immunostimulatory lipid may be a glycolipid. Suitable immunostimulatory glycolipids will be familiar to persons skilled in the art, illustrative examples of which are described in Kim et al. (Expert Rev Vaccines. 2008; 7(10): 1519-1532), Godfrey, et al. (Nat. Immunol 2015; 16: 1114-1123, and Cerundolo, et al. (Nat. Rev. Immunol. 2009; 9: 28-38). In an embodiment, the glycolipid is an alpha-anomeric glycolipid or a beta-anomeric glycolipid. In an embodiment, the glycolipid is an alpha-anomeric glycolipid. In an embodiment, the alpha-anomeric glycolipid is an alpha-anomeric glycosphingolipid. In an embodiment, the alpha-anomeric glycosphingolipid is α-galactosylceramide or α-glucosylceramide. In another embodiment, the alpha-anomeric glycosphingolipid is α-glucosylceramide. In another embodiment, the glycolipid is a beta-anomeric glycolipid. In another embodiment, the beta-anomeric glycolipid is a beta-anomeric glycosphingolipid. In another embodiment, the beta-anomeric glycolipid is selected from the group consisting of β-mannosylceramide, β-glucosylceramide and β-galactosylceramide. In another embodiment, the beta-anomeric glycolipid is β-mannosylceramide.

The immunostimulatory lipid may be a phospholipid. Suitable immunostimulatory phospholipids will be familiar to persons skilled in the art, illustrative examples of which are described in Godfrey, et al. (2015; ibid). In an embodiment, the phospholipid is lyso-phosphatidylcholine or lysophosphatidyl-ethanolamine. Immunostimulatory lipids, as described herein, including immunostimulatory glycolipids and phospholipids, can vary in the length and saturation of their fatty acid chains (including the acyl and/or sphingosine chains). These are typically referred to by reference to the number of carbons on the acyl chain (e.g., α-GalCer C26, α-GalCer C24, α-GalCer C20:2 etc). For instance, α-GalCer, also known as KRN7000, has an 18C phytosphingosine and a 26C acyl chain, whereas α-GalCer C20:2 has a C20 acyl chain and cis-diunsaturation at C11 and C14. Suitable immunostimulatory lipids of varying length and saturation of their fatty acid chains will be familiar to persons skilled in the art, illustrative examples are described in Wun et al. (2011. Immunity 34: 327-339). Such variation in the length and saturation of their fatty acid chains may impact on the immunostimulatory capacity of such lipids. Nevertheless, it is to be understood that the immunostimulatory lipids described herein, when administered to a host with an immunogen (e.g., by enhancing the update of the VLP/polynucleotides, as described herein, by antigen presenting cells), will be capable of potentiating the host's immune response to the immunogen, irrespective of the length and saturation of their fatty acid chains. Methods of determining whether an immunostimulatory lipid is capable of potentiating the host's immune response to an immunogen according to the methods described herein, irrespective of the length and saturation of their fatty acid chains, will be known to persons skilled in the art. Where two or more immunostimulatory or adjuvanting lipids are contemplated, it is generally desirable to select a combination of two or immunostimulatory or adjuvanting lipids that further enhance or potentiate the host's immune response to the immunogen, irrespective of whether the enhancement or potentiation is additive or synergistic. The present disclosure also contemplates the use of one or more non-immunostimulatory lipids (e.g., neutral, cationic and/or ionisable lipids, cholesterol and polyethylene glycol). Illustrative examples of suitable lipids are also described in US 20190314291, the entire contents of which is incorporated herein by reference.

The terms “immunisation” and “vaccination” are used interchangeably herein to refer to the administration of the vaccine construct and/or VLP, as described herein, to a subject for the purposes of raising an immune response and can have a prophylactic effect, a therapeutic effect, or a combination thereof.

As used herein, the terms “treat,” “treated,” or “treating” when used with respect to a disease or pathogen refers to a treatment which increases the resistance of a subject to the disease or to infection with a pathogen (i.e. decreases the likelihood that the subject will contract the disease or become infected with the pathogen), as well as a treatment after the subject has contracted the disease or become infected with the pathogen in order to fight a disease or infection (e.g., to reduce, eliminate, ameliorate or otherwise stabilise a disease or infection). In an embodiment, the vaccine construct and/or VLP, as described herein, are capable of providing protective immunity to a host. The term “protective immunity,” as used herein, is intended to mean the ability of a host (e.g., salmon), to resist (delayed onset of symptoms, reduced severity of symptoms or lack of symptoms), as a result of its exposure to the vaccine construct and/or VLP, as described herein, disease or death that would otherwise follow exposure to a pathogen. Protective immunity is typically achieved by one or more of mucosal, humoral, or cellular immunity.

The term “subject” or “host” refers to an animal in need of treatment. Preferably, the subject or host is a fish, more preferably salmon, including both fresh water and salt water species.

The present disclosure also extends to pharmaceutical kits or packs comprising the vaccine construct and/or VLP and/or compositions, as described herein, including for use as a vaccine. Individual components of the kit can be packaged in separate containers, associated with which, when applicable, can be a notice in the form prescribed by a governmental agency regulating the manufacture, use or sale of pharmaceuticals or biological products, which notice reflects approval by the agency of manufacture, use or sale for administration. The kits may optionally further comprise one or more other therapeutic agents for use in combination with the vaccine construct and/or VLP and/or compositions, as described herein. The kit may optionally contain instructions or directions outlining the method of use or administration regimen. When one or more components of the kit are provided as solutions, for example an aqueous solution, or a sterile aqueous solution, the container means may itself be an inhalant, syringe, pipette, eye dropper, or other such like apparatus, from which the solution may be administered to a subject or applied to and mixed with the other components of the kit. The components of the kit may also suitably be provided in dried or lyophilised form and the kit can additionally contain a suitable solvent for reconstitution of the lyophilised components. Irrespective of the number or type of containers, the kits of the invention also may comprise an instrument for assisting with the administration of the composition to a patient. Such an instrument may be an inhalant, syringe, pipette, forceps, measured spoon, eye dropper or similar medically approved delivery vehicle.

In order that the invention may be readily understood and put into practical effect, particular preferred embodiments will now be described by way of the following non-limiting examples.

### Examples

### Materials and Methods

### Cohabitation Challenge Model Trial Design

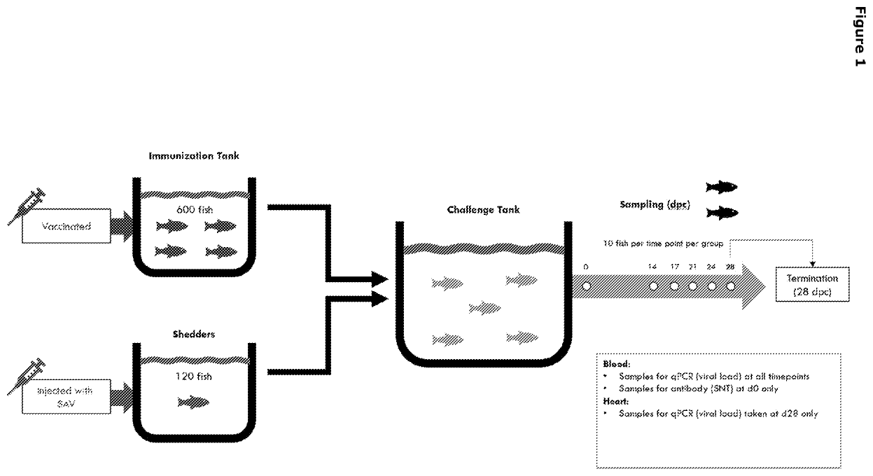

FIG. 1 is schematic describing the cohabitation challenge model trial design.

The fish in the trial were Atlantic salmon (Salmo salar), StofnFiskur Optimal strain. The fish were 24.2 g average weight at vaccination. The fish were maintained in freshwater, adjusted according to minimum 70% oxygen in effluent water, with the maximum stocking density of 40 kg/m₃, at 12 °C±1 °C. The fish were fed 1-2% of biomass by automatic feeder. See table below for summary of hatchery conditions.

| | |
|---|---|
| Salinity | 0% (fresh water) |
| Stocking density | Max 40 kg/m³ |
| Temperature | 12 °C ± 1°C |
| Flow | Adjusted according to minimum 70% |
| | oxygen in effluent water |
| Water discharge | Tube overflow system |
| Cleaning | Once a day |
| Photoperiod regime | Light (L):Dark (D) = 24:0 |
| Feeding | 1-2% of biomass by automatic feeder |

The fish were acclimatised at the test facility for a minimum of one week prior to vaccination, and were starved for a minimum of 48 hours prior to vaccination. The fish were identified by visible implant elastomer (VIE) tags, unvaccinated at parr stage, and kept in freshwater throughout the trial. Vaccination was performed by intramuscular (i.m.) injection (0.05 mL/fish), using 1 mL disposable syringes supplied with 0.5×3 mm stainless steel needles positioned at 90° on the left epaxial muscle, central to the dorsal fm and above the midline. Duration of immunisation was 500-degree days. During the immunisation period, vaccinated fish were kept in a single tank. Shedders (SAV negative fish which are experimentally infected with the SAV challenge virus and mixed with the test groups at an appropriate ratio in order to provide an authentic source of infectious virus) were kept in another tank to minimise handling at challenge.

After immunisation, the fish were challenged with SAV3 using a well established cohabitation challenge model. An illustrative example of suitable cohabitation challenge models is described in Graham et al (2011, J Fish Dis 34: 273-286), the contents of which are incorporated herein by reference in their entirety. Shedders were anaesthetised, injected intraperitoneally (i.p.) with 0.1 mL thawed dose adjusted SAV3 (titre ca. 2×10³ TCID₅₀/fish) solution. The shedders were marked by adipose fm clipping, and starved for a minimum of 24 hours prior to challenge. The i.p. injected shedders were introduced to the tank with the vaccinated fish.

The SAV3 was a Norwegian isolate from a field outbreak in 2007, grown in CHSE-2014 at Norwegian Veterinary Institute. Original concentration 10⁵⁻⁵TCID₅₀/mL. Challenge inoculum was diluted with PBS in 1:5 ratio. Published: Taksdal T et al., J Fish Dis 2015 (12):1047-61.

Duration the challenge was 28 days. The first day of vaccination is day 0. Samplings were carried out at 0, 14, 17, 21, 24 and 28 days post-challenge (dpc). At each sampling point, 10 randomly selected fish from each group were sampled.

Fish at the terminal stage were taken out of the study and killed by an overdose of anaesthetic and/or by a lethal blow to the head. Culled fish were equated with dead fish in subsequent efficacy calculations and reporting. Only fish that were not able to self-propel or maintain their position in the water, and/or fish lying on the floor of the trial tank or were not able to return to an upright position when prodded or turned on their side were culled.

At the termination of challenge, 5 randomly selected shedders were sampled.

No mortality occurred during this trial, therefore no mortalities were sampled during this trial.

### Test Validity and Total Mortality

Heart tissue was sampled from five randomly selected shedders at the termination of challenge for qPCR analysis to verify presence of SAV infection.

### Sampling and Analysis

Blood: Following the sampling schedule described in FIG. 1, ten fish were randomly sampled from each group and heparinised blood was obtained using vacutainer tubes and needles. For SAV qPCR testing, a whole blood sample and a white blood cell pellet sample was obtained for each fish. For the determination of SAV neutralising antibodies, plasma samples were prepared. All samples were stored at −80 °C prior to shipping to the appropriate analytical laboratory, namely Patogen A S for qPCR analysis and Norwegian Veterinary Institute for the SAV neutralising antibody analysis.

### Quantitative Analysis of SAV RNA

A quantitative polymerase chain reaction (qPCR) assay was used to quantify SAV RNA levels in whole blood and heart tissue post-virus challenge in the fish of this study. The qPCR analysis of all samples was carried out using a validated and ISO17025 accredited method by Patogen A S (Patogen A S, Ålesund, Norway), as previously described by Hodneland, K and Endresen, C (2006); J. Virol. Methods 131 (2) 184-192), the entire contents of which is incorporated herein by reference. The cut off Ct-value was set to 37. Ct-values were converted to SAV RNA copy number by Patogen.

### SAV Neutralising Antibody

The assay was performed at Norwegian Veterinary Institute according to the general method of Graham et al, 2003 with some modifications. In short, plasma samples were titrated against SAV3 (Isolate 4 from Taksdal et al., 2015) or SAV2 (Isolate 1 from Taksdal et al., 2015, GenBank ref. HE863664) on CHSE-214 cells in 2 parallel wells (96 well plate) in a two-fold dilution series. After 7 days of incubation at 15 °C, the cell layer was fixed with 80% acetone. SAV-infected cells were then visualised in an indirect immuno-fluorescence test according to the procedure described by Falk, Namork & Dannevig, 1998, but with the use of monoclonal antibody 17H23 directed against the E2 glycoprotein of SAV (Merour et al. 2016) as the primary antibody and with biotin labelled goat anti-mouse Ig and FITC-labelled streptavidin included in the assay. Positive staining was scored in a fluorescence microscope. Antibody titres≥1:20 were recorded as positive results.

### Statistical Analysis

For the SAV viraemia data (Genome Copy Number; GCN) described in Examples 1 and 2, Log10 distributions were used in non-parametric models and testing, using the negative control group dbDNA eGFP 7.5 μg and day 0 as a reference comparison (AUCLogGCN). The NPAR1WAY procedure was used which performs nonparametric tests for location and scale differences across a one-way classification. Wilcoxon scores (Rank Sums) were calculated for groups for outcome variables. Pairwise two-sided multiple comparison analysis was performed using the Dwass, Steel, Critchlow-Fligner (DSCF) Method.

For the SAV neutralizing antibody data described in Examples 3 and 4. data were analysed using three complimentary methods. The primary test was the non-parametric two-sided Jonkheere-Terpstra statistical test of trend, supported by Fishers Exact Test and Wilcoxon Score.

### Example 1: Closed Linear Polynucleotide is Effective as a Vehicle for Immunsing Atlantic Salmon Against SAV

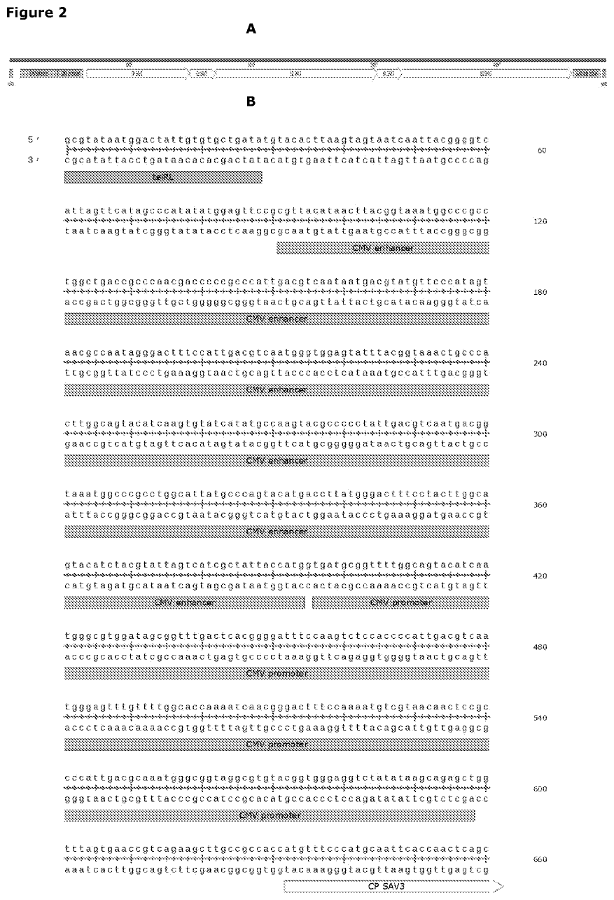

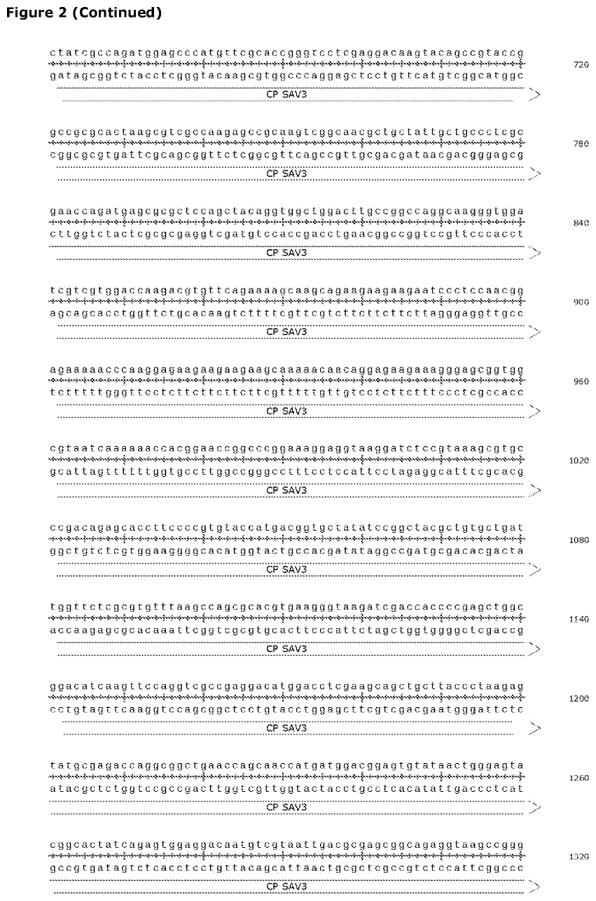

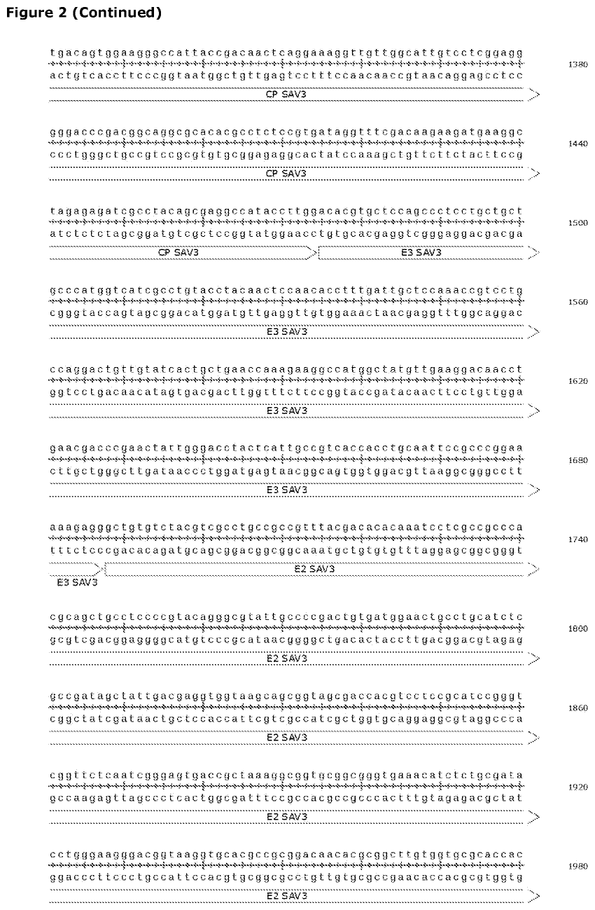

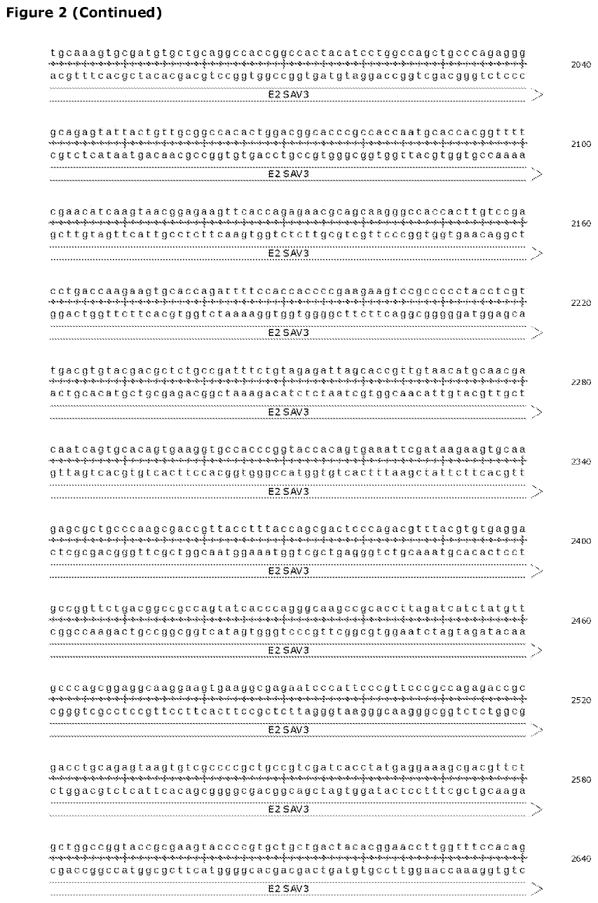

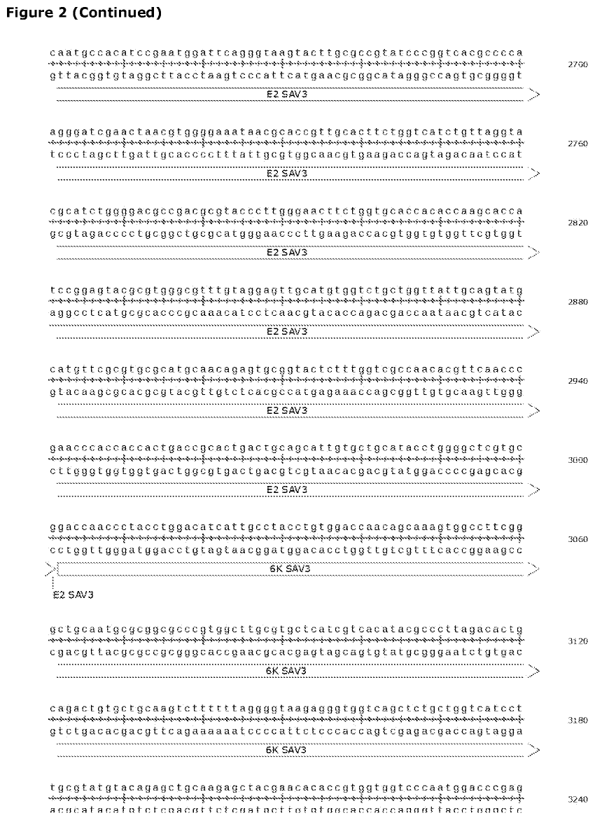

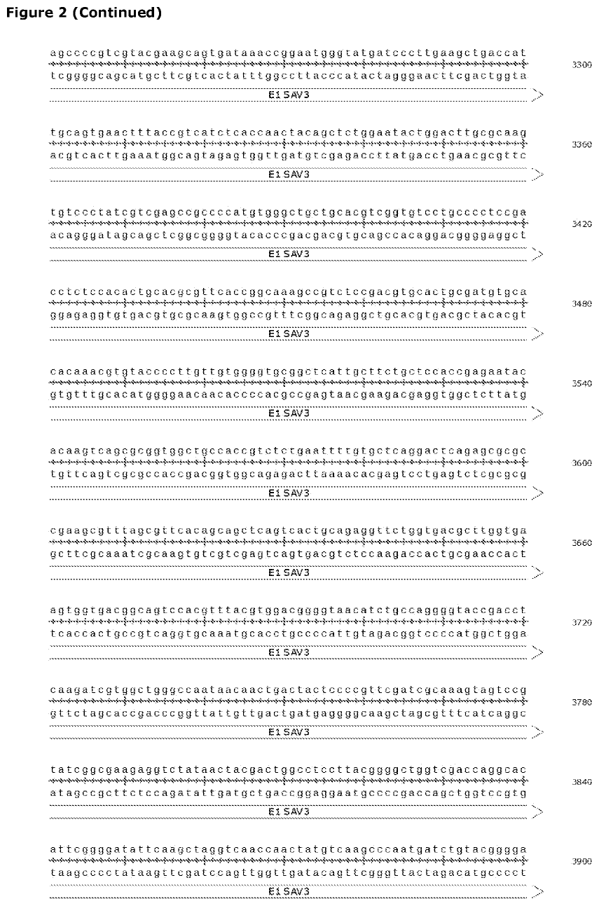

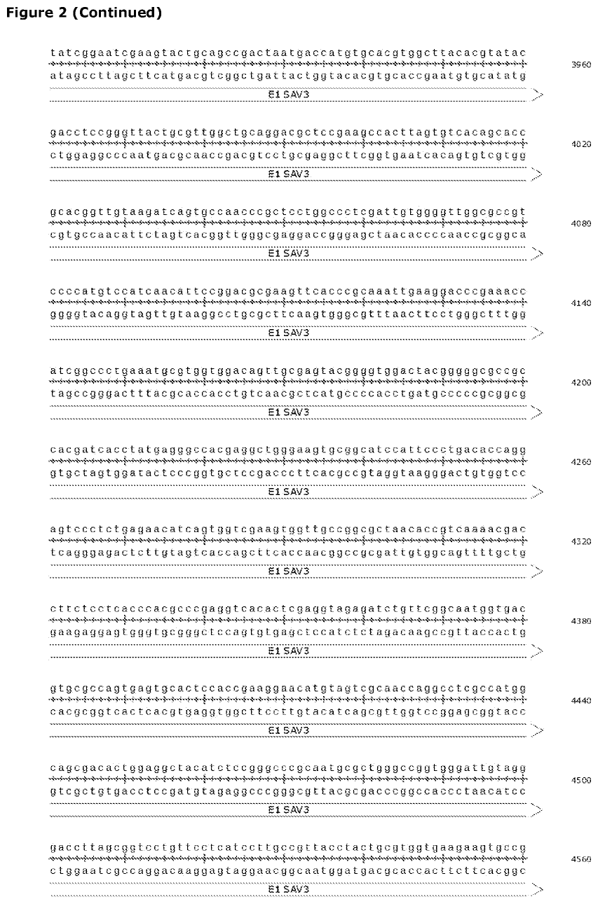

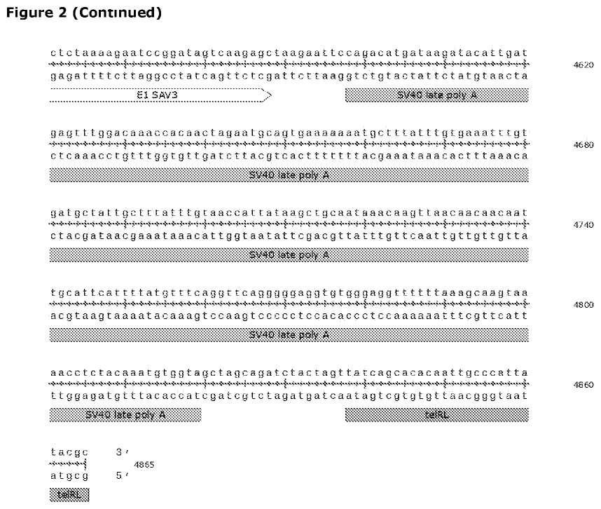

Plasmid DNA have been the predominant DNA-based immunisation vectors. To investigate whether closed linear polynucleotides would be useful for immunising Atlantic salmon, a closed linear polynucleotide construct comprising a polynucleotide sequence comprising SAV3 capsid, E3, E2, 6K and E1 nucleic acid sequences (SEQ ID NO:2) was generated (dbDNA SAV3 in FIG. 2). Doggybone DNA (dbDNA™) is a proprietary synthetic closed linear double-stranded DNA construct. Construct 3-dbDNA SAV3 (SEQ ID NO:14) was used to immunise fish, and its effects compared to plasmid DNA-based ClyNav® vaccine using a cohabitation challenge model trial design. A dbDNA comprising polynucleotide GFP sequences (dbDNA eGFP) was used as a negative control.

Fish were immunised with:
7.5 μg of 3-dbDNA SAV3;
7.5 μg of dbDNA eGFP (negative control); or
“on-label”/recommended dose of ClyNav® (6.0-9.4 μg plasmid DNA per 0.05 ml dose).

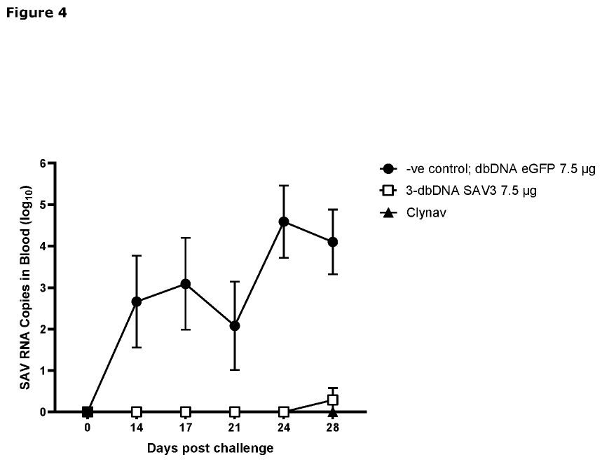

As expected, fish receiving dbDNA eGFP showed high levels of SAV3 genome copy number/mL blood (FIG. 4). Fish receiving 7.5 μg of dbDNA SAV3 or ClyNav® showed low viral titres throughout the study sampling period (i.e., up to 28 days).

This indicates that dbDNA™ is at least as capable as plasmid DNA-based vehicle as inducing a protective immune response. Further, this shows that dbDNA™ construct comprising SAV3 nucleic acid sequences may be useful for immunising Atlantic salmon.

### Example 2: Modified Dbdna SAV3 Construct Increases Potency

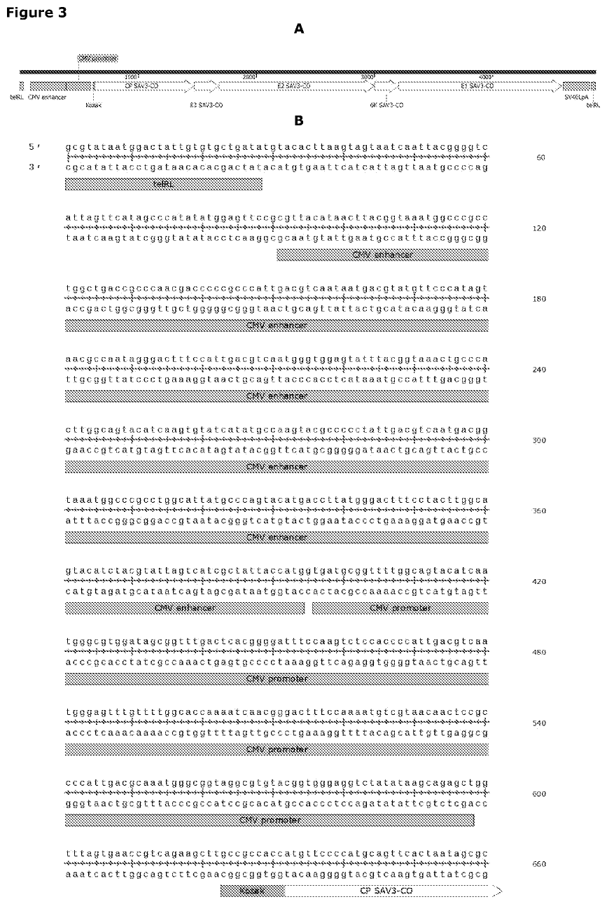

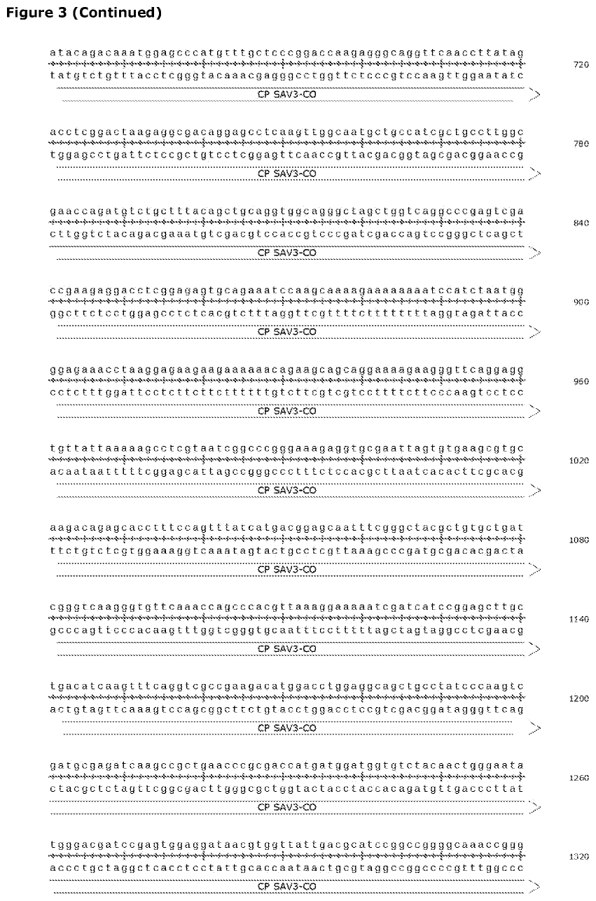

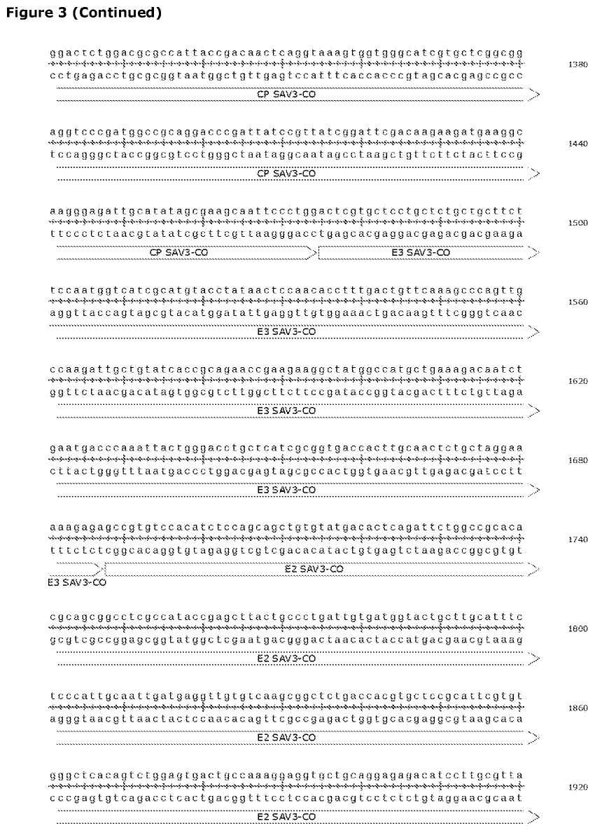

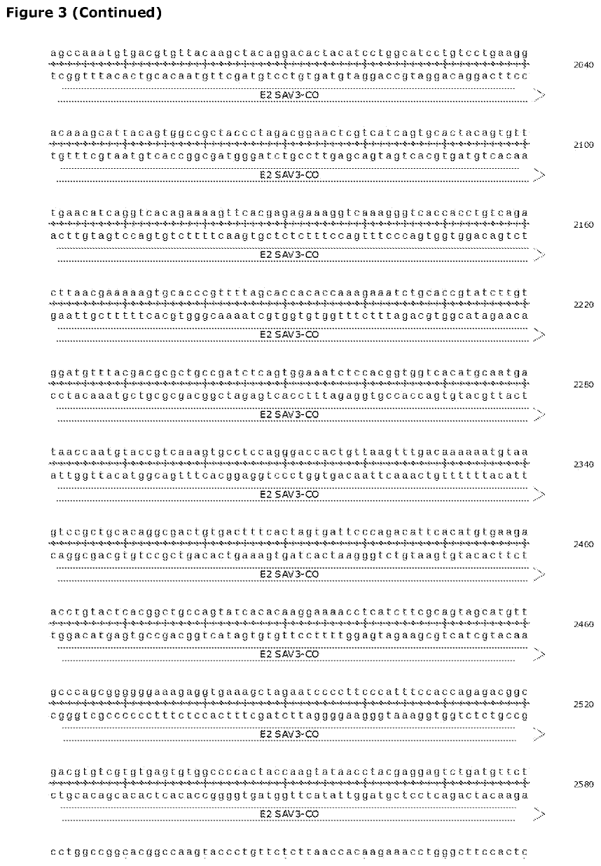

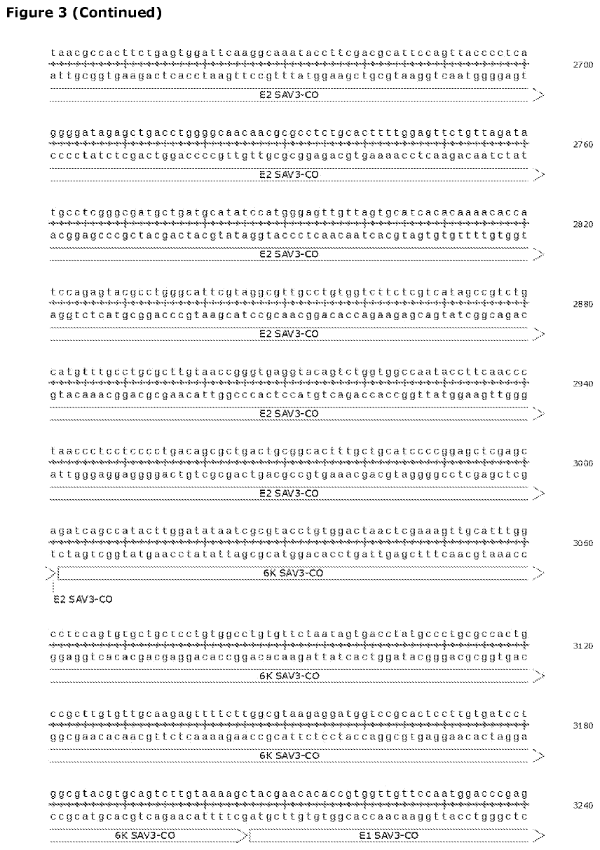

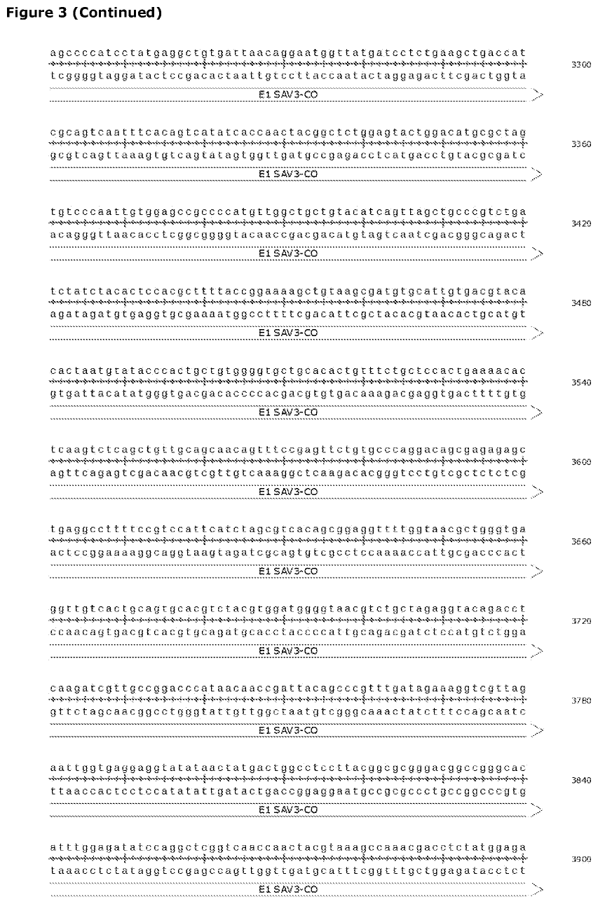

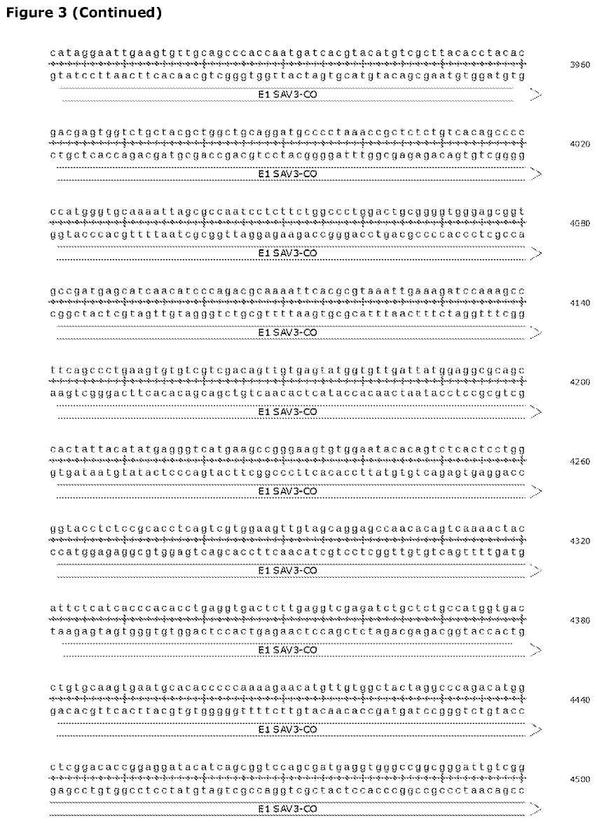

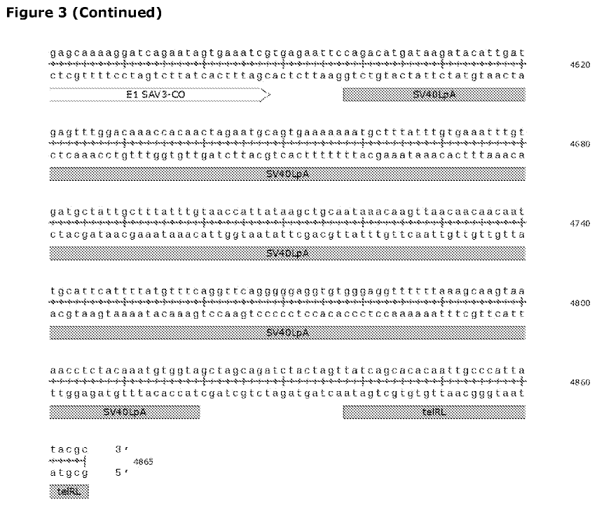

SEQ ID NO:2 is the wildtype polynucleotide sequence (GenBank: AY604238) that encodes for a SAV subtype-3 structural polyprotein having the amino acid sequence of SEQ ID NO:1. The polynucleotide sequence encoding for SEQ ID NO:1 was modified to comprise codons that were optimised for expression in fish cells. SEQ ID NO: 3 is one such codon-optimised nucleotide sequence that was inserted into a closed linear proprietary synthetic closed linear double-stranded DNA construct to generate 5-dbDNA SAV3-CO construct (SEQ ID NO:15), as shown in FIG. 3.

Fish were immunised using 1 (very low dose), 2.5 (low dose) or 7.5 (reference dose) μg dose of either 3-dbDNA SAV3 or 5-dbDNA SAV3-CO. As a negative control, a 7.5 μg dose of dbDNA eGFP was used. The number of fish (out of the ten fish sampled) that tested positive for SAV (detection of SAV in blood samples) at each timepoint is shown in the Table 2 below:

TABLE 2
Detection of SAV in fish receiving different
doses of 3-dbDNA SAV3 or 5-dbDNA SAV3-CO

| Time | dbDNA | | | | | | |
|---|---|---|---|---|---|---|---|
| point | eGFP | 5-dbDNA SAV3-CO | | | 3-dbDNA SAV3 | | |
| (days) | 7.5 μg | 7.5 μg | 2.5 μg | 1 μg | 7.5 μg | 2.5 μg | 1 μg |
| 0 | 0 (0%) | 0 (0%) | 0 (0%) | 0 (0%) | 0 (0%) | 0 (0%) | 0 (0%) |
| 14 | 4 (40%) | 0 (0%) | 0 (0%) | 0 (0%) | 0 (0%) | 0 (0%) | 1 (10%) |
| 17 | 5 (50%) | 0 (0%) | 0 (0%) | 1 (10%) | 0 (0%) | 0 (0%) | 4 (40%) |
| 21 | 3 (30%) | 0 (0%) | 0 (0%) | 1 (10%) | 0 (0%) | 2 (20%) | 5 (50%) |
| 24 | 8 (80%) | 0 (0%) | 1 (10%) | 0 (0%) | 0 (0%) | 0 (0%) | 2 (20%) |
| 28 | 8 (80%) | 0 (0%) | 2 (20%) | 2 (20%) | 1 10%) | 1 (10%) | 1 (10%) |
| Total | 28 | 0 | 3 | 4 | 1 | 3 | 13 |
| positive | | | | | | | |
| (out of 60 | | | | | | | |
| sampled) | | | | | | | |

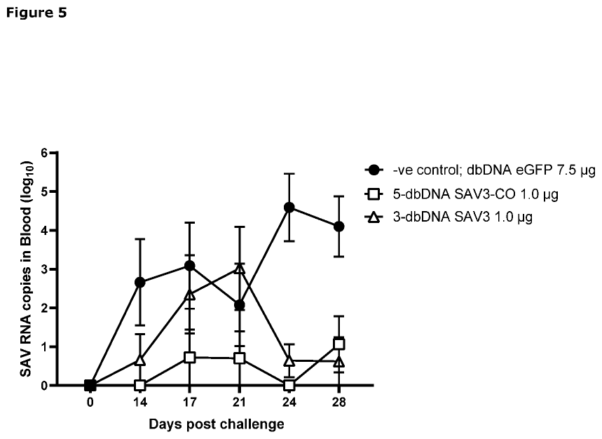

It is notable that the codon-optimisation resulted in a significant increase in potency, as seen by the decrease in the number of fish testing positive for SAV in the cohort receiving 1 μg of the codon optimised 5-dbDNA SAV3-CO, when compared to 1 μg of 3-dbDNA SAV3. FIG. 5 shows that the viraemia (SAV genome copy number) observed in the cohort receiving 1 μg 3-dbDNA SAV3 is longer in duration and greater in magnitude when compared to the cohort receiving 1 μg of the codon optimised 5-dbDNA SAV3-CO. Non-parametric statistical analysis AUCLogGCN indicates that the study deviates from null hypothesis that there was no effect of vaccination on AUCLogGCN in fish (P<0,01).

Pairwise group non-parametric comparison of AUCLogGCN indicates the overall level of viraemia observed in fish vaccinated with 3-dbDNA SAV3 1.0 μg was not significantly lower than that seen in the negative control group (i.e. there was no meaningful impact of vaccination). In contrast, the overall level of viraemia seen for 5-db SAV3-CO 1.0 μg was significantly lower than that seen in the negative control group, indicating a positive impact of vaccination with the codon optimized form, even at very low dose (1.0 μg).

Overall, the data show that a codon-optimised polynucleotide sequence encoding a SAV3 VLP (5-dbDNA SAV3-CO) is effective at the doses tested and may have significant dose-sparing potential when compared to non-optimal sequences.

### Example 3: Modified Dbdna SAV3 Construct Provides Improved Neutralising Antibody Response

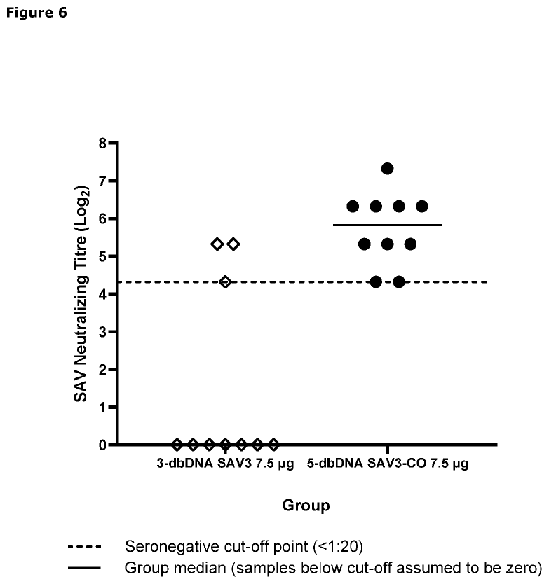

The neutralising antibody response in fish receiving the SAV constructs was examined at 500-degree days post-immunisation, as described above. The results are shown in FIG. 6. Fish receiving the reference dose of codon-optimised 5-dbDNA SAV3-CO had significantly higher levels of neutralising antibody titres when compared to fish receiving the equivalent dose of 3-dbDNA SAV3 (not codon-optimised). The non-parametric two-sided Jonkheere-Terpstra statistical test of trend indicated that titres in fish vaccinated with 3-dbDNA SAV3 group were significantly more shifted to lower scores compared to fish vaccinated with 5-dbDNA SAV3-CO 7.5 μg (JT P-value=<0.01). The binary classification of negative (<1:20) versus positive titres (≥1:20) indicated fish in the 3-dbDNA SAV3 group had significantly more negative titres than the 5-dbDNA SAV3-CO 7.5 μg group (Fisher Exact P-value<0.01). The Wilcoxon Scores of the serum titre categories were significantly lower in the 3-dbDNA SAV3 group than in the 5-dbDNA SAV3-CO 7.5 μg group (P-value<0.01).

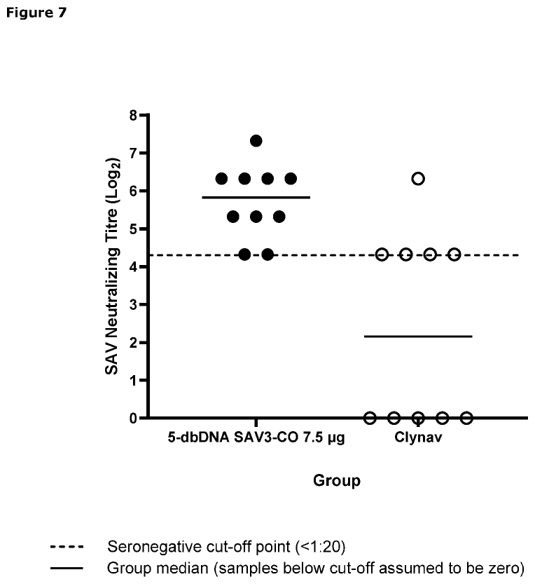

Furthermore, the reference dose of codon-optimised 5-dbDNA SAV3-CO provided a higher neutralising antibody when compared to the recommended dose of the non-codon optimized plasmid DNA vaccine ClyNav®, as seen in FIG. 7. Importantly, only half the fish in the ClyNav® group seroconverted (i.e. produced neutralising antibodies) following vaccination, versus 100% of the 5-dbDNA SAV3-CO fish. The non-parametric two-sided Jonkheere-Terpstra statistical test of trend indicated that titres in fish vaccinated with ClyNav® were significantly more shifted to lower scores compared to fish vaccinated with 5-dbDNA SAV3-CO at 7.5 μg per dose (JT P-value=<0.01). The binary classification of negative (<1:20) versus positive titres (1:20) indicated fish in the ClyNav® group had significantly more negative titres than the 5-dbDNA SAV3-CO 7.5 μg group (Fisher Exact P-value=0.03). The Wilcoxon Scores of the serum titre categories were significantly lower in the ClyNav® group than in 5-dbDNA SAV3-CO 7.5 μg group (P-value<0.01).

### Example 4: Modified Dbdna SAV3 Construct Provides Potent Cross-Neutralisation of SAV2

To examine if the immune response induced by the codon optimised 5-dbDNA SAV3-CO would also protect against SAV2 (another SAV sub-group that circulates in Europe), the neutralisation assay was also performed with SAV2.

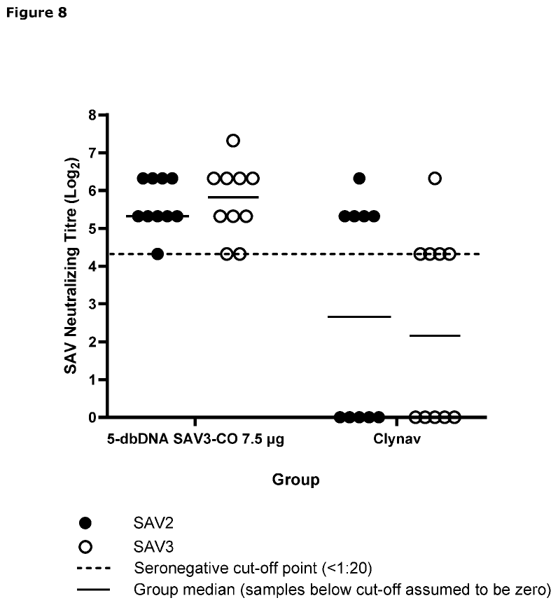

The data in FIG. 8 demonstrate that 5-dbDNA SAV3-CO not only provided a cross-neutralising antibody response of SAV2, but SAV2 neutralising titres were again detected in 100% of the fish compared with only 50% of the fish receiving the recommended dose of ClyNav®. The non-parametric two-sided Jonkheere-Terpstra statistical test of trend indicated that titres in fish vaccinated with ClyNav® were significantly more shifted to lower scores compared to fish vaccinated with 5-dbDNA SAV3-CO at 7.5 μg per dose (JT P-value=0.02). The binary classification of negative (<1:20) versus positive titres (1:20) indicated fish in the ClyNav® group had significantly more negative titres than the 5-dbDNA SAV3-CO 7.5 μg group (Fisher Exact P-value=0.03). The Wilcoxon Scores of the serum titre categories were significantly lower in the ClyNav® group than in the 5-dbDNA SAV3-CO 7.5 μg group (P-value=0.04).

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

1. A vaccine construct comprising a polynucleotide encoding a salmon alphavirus-like particle, wherein the polynucleotide comprises:
(a) a nucleic acid sequence encoding a salmon alphavirus capsid protein; and
(b) at least one nucleic acid sequence encoding a salmon alphavirus envelope protein selected from the group consisting of an E3, E2, 6K and E1 envelope protein;
wherein the nucleic acid sequence encoding the salmon alphavirus capsid protein and/or the at least one nucleic acid sequence encoding the salmon alphavirus envelope protein is codon-optimised to resemble codon usage in a fish cell, and wherein the polynucleotide provides an enhanced immune response against salmon alphavirus when compared to a polynucleotide encoding a salmon alphavirus-like particle in which the nucleic acid sequences are not codon-optimised.

2. The vaccine construct of claim 1, wherein the polynucleotide comprises:
(a) a nucleic acid sequence encoding a salmon alphavirus capsid protein, wherein the nucleic acid sequence encoding the salmon alphavirus capsid protein comprises the nucleic acid sequence set forth in SEQ ID NO: 5 or a nucleic acid sequence having at least 76.5% sequence identity thereto; and
(b) at least one nucleic acid sequence encoding a salmon alphavirus envelope protein selected from the group consisting of an E3, E2, 6K and E1 envelope protein, wherein:
i. the nucleic acid sequence encoding the E3 envelope protein comprises the nucleic acid sequence set forth in SEQ ID NO:7 or a nucleic acid sequence having at least 80.5% sequence identity thereto;
ii. the nucleic acid sequence encoding the E2 envelope protein comprises the nucleic acid sequence set forth in SEQ ID NO:9 or a nucleic acid sequence having at least 74.5% sequence identity thereto;
iii. the nucleic acid sequence encoding the 6K envelope protein comprises the nucleic acid sequence set forth in SEQ ID NO: 11 or a nucleic acid sequence having at least 73% sequence identity thereto; and
iv. the nucleic acid sequence encoding the E1 envelope protein comprises the nucleic acid sequence set forth in SEQ ID NO: 13 or a nucleic acid sequence having at least 76.5% sequence identity thereto.

3.-4. (canceled)

5. The vaccine construct of claim 1, wherein the polynucleotide comprises the nucleic acid sequence set forth in SEQ ID NO:3 or a nucleic acid sequence having 76% sequence identity thereto.

6. (canceled)

7. The vaccine construct of claim 1, wherein the nucleic acid sequence encoding the salmon alphavirus capsid protein or the salmon alphavirus envelope protein is operatively linked to a CMV promoter or an EF1α promotor.

8.-10. (canceled)

11. The vaccine construct of claim 1, further comprising a SV40 poly(A) sequence.

12. The vaccine construct of claim 1, further comprising telomeric sequences that flank both ends of the polynucleotide construct.

13. (canceled)

14. The vaccine construct of claim 1, wherein the polynucleotide construct is a closed linear polynucleotide.

15.-17. (canceled)

18. A method for treating or protecting against salmon alphavirus infection in a subject, the method comprising administering to a subject in need thereof the vaccine construct of claim 1.

19. The method of claim 18, wherein the vaccine construct is administered to the subject by intramuscular injection.

20. The method of claim 19, comprising administering to the subject the vaccine construct in an amount of from about 0.5 micrograms to about 10 micrograms.

21. The method of claim 18, wherein the subject has, or is at risk of developing, salmonoid pancreatic disease.

22.-23. (canceled)

24. A method of inducing an immune response against salmonid alphavirus in a subject, the method comprising administering to a subject in need thereof the vaccine construct of claim 1.

25. The method of claim 24, wherein the salmonid alphavirus is a SAV2 subtype.

26. The method of claim 24, wherein the salmonid alphavirus is a SAV3 subtype.

27. The method of claim 24, wherein the immune response comprises an antibody response, wherein the antibodies are capable of binding specifically to and neutralising the salmon alphavirus.

28. The method of claim 27, wherein the antibodies are capable of binding specifically to and neutralising SAV2 and SAV3 subtypes.

29. The method of claim 24, wherein the vaccine construct, the salmon alphavirus-like particle or the composition is administered to the subject by intramuscular injection.
